SEQUENCE LISTING (11 sequences)

SEQ ID NO: 1 - protein, 120 aa, synthetic construct
EVQLVESGGGLVQPGGSLRLSCAASGSFFRIVAMGWYRQAPGSQRELVATITRGGSTYYADSVKGRFTISRDNAKNTLYLQMNSLRAEDTAVYYCNVRVEVPFMQPNDYWGQGTLVTVSS

SEQ ID NO: 2 - protein, 8 aa, synthetic construct
GSFFRIVA

SEQ ID NO: 3 - protein, 7 aa, synthetic construct
ITRGGST

SEQ ID NO: 4 - protein, 14 aa, synthetic construct
NVRVEVPFMQPNDY

SEQ ID NO: 5 - protein, 119 aa, synthetic construct
QVQLLESGGGLVQPGGSLRLSCAASGFGFGNSLMSWVRRAPGKGLEWVSTIYPRGSFTDYADSVKGRFTISRDNSRNTLYLQMNSLRAEDTAVYYCATGWGRSSDYDPPGQGTLVTVSS

SEQ ID NO: 6 - protein, 10 aa, synthetic construct
GFGFGNSLMS

SEQ ID NO: 7 - protein, 9 aa, synthetic construct
YPRGSFTDY

SEQ ID NO: 8 - protein, 11 aa, synthetic construct
TGWGRSSDYDP

SEQ ID NO: 9 - protein, 8 aa, synthetic construct
GFGFGNSL

SEQ ID NO: 10 - protein, 8 aa, synthetic construct
IYPRGSFT

SEQ ID NO: 11 - protein, 12 aa, synthetic construct
ATGWGRSSDYDP

(19) United States
(12) Patent Application Publication
(10) Pub. No.: US 2026/0216366 A1
(43) Pub. Date: Jul. 30, 2026

(54) CONJUGATE AND USES THEREOF

(71) Applicant: Full-Life Technologies UK Limited, London (GB)

(72) Inventors: Xiaozhu WANG, Shanghai (CN); Chung Lim WONG, Shanghai (CN)

(21) Appl. No.: 19/143,228

(22) PCT Filed: Dec. 26, 2023
(86) PCT No.: PCT/CN2023/141919

§ 371 Date: Jun. 25, 2025

(30) Foreign Application Priority Data
Dec. 26, 2022 (WO) PCT/CN2022/141998

(51) Int. Cl. A61K 47/68; A61K 47/54; A61K 47/60; A61K 47/69; A61K 51/10; A61P 35/00

(52) U.S. Cl. CPC A61K 47/6889; A61K 47/545; A61K 47/60; A61K 47/6813; A61K 47/6815; A61K 47/6851; A61K 47/6863; A61K 47/6929; A61K 51/1045; A61K 51/1063; A61K 51/1096; A61P 35/00

(57) ABSTRACT

A conjugate comprises: (a) a single-domain antibody (sdAb) that specifically binds to a tumor antigen, (b) a drug, and (c) a functional linker which links the drug to the sdAb. The use of the conjugate is treating cancer. The method for preparing the conjugate comprises conjugating the functional linker and the drug to the sdAb.

### RELATED APPLICATIONS

The present application claims priority of International Application No. PCT/CN2022/141998, filed on Dec. 26, 2022, the disclosure of which is hereby incorporated by reference in its entirety.

### FIELD OF THE INVENTION

This disclosure relates to a novel conjugate comprising: (a) a single-domain antibody (sdAb) that specifically binds to a tumor antigen, (b) a drug, and (c) a functional linker which links the drug to the single-domain antibody; and uses of the conjugate for treating cancer. Also provided is a method for preparing the conjugate.

### BACKGROUND

Antibody-drug conjugate is one of the most promising therapeutic approaches in the field of oncology. As a targeted therapy, antibody-drug conjugate (ADC) use an antibody to selectively deliver a cytotoxic drug to a target, such as a tumor-associated antigen, and have achieved significant clinical and commercial successes. There are several factors contributing to the overall efficacy of ADC therapies including tumor penetration and accumulation, target binding and cellular uptake, release of active catabolic products within the target cells, as well as the pharmacokinetic (PK) profile of the conjugates. Similar to ADC, radionuclide drug conjugate (RDC) uses antibody or small molecule to precisely deliver cytotoxic/imaging radioisotope payload to a target, which could potentially increase the efficacy and prevent toxicity due to systemic exposure of the radioisotope. Previous studies showed that a full-length antibody specifically binding to a tumor antigen is capable of carrying a radioisotope to the tumor tissue. However, due to its large size, RDC carried by a full-length antibody still could cause long systemic retention of the radioisotope, resulting in undesired toxicity.

A single-domain antibody (sdAb), also known as a nanobody, is an antibody fragment consisting of a single monomeric variable antibody domain. Like a full-length antibody, it is capable of binding selectively to a specific antigen but has a smaller molecular weight of only 12-15 kDa. However, the small size causes sdAb tends to be trapped in kidney, which could lead to renal toxicity and insufficient efficacy towards the target tissue (e.g., the tumor) in therapeutic applications. Therefore, improved target therapy is in demand in the field.

### BRIEF SUMMARY OF THE INVENTION

In one aspect, provided is a conjugate comprising: (a) a single-domain antibody (sdAb) that specifically binds to a tumor antigen; (b) a drug; and (c) a functional linker comprising a functional moiety which links the drug to the sdAb, wherein the functional linker increases tumor uptake, accumulation, and/or retention of the drug when the conjugate is administered to a subject compared to a conjugate without the functional moiety.

In one aspect, provided is a conjugate comprising: (a) a single-domain antibody (sdAb) that specifically binds to a tumor antigen; (b) a drug; and (c) a functional linker comprising a functional moiety which links the drug to the sdAb, wherein the functional linker decreases kidney uptake, accumulation, and/or retention of the drug when the conjugate is administered to a subject compared to a conjugate without the functional moiety.

In another aspect, provided is a conjugate comprising: (a) a single-domain antibody (sdAb) that specifically binds to a tumor antigen; (b) a drug; and (c) a functional linker comprising a functional moiety which links the drug to the sdAb, wherein the functional linker increases tumor/kidney ratio of the drug when the conjugate is administered to a subject compared to a conjugate without the functional moiety.

In another aspect, provided is a conjugate comprising: (a) a single-domain antibody (sdAb) that specifically binds to a tumor antigen; (b) a drug; and (c) a functional linker comprising a functional moiety which links the drug to the sdAb, wherein the functional linker prolongs blood circulation and/or half-life of the drug when the conjugate is administered to a subject compared to a conjugate without the functional moiety.

In another aspect, provided is a conjugate comprising: (a) an antibody mimetic that specifically binds to a tumor antigen, wherein the antibody mimetic is selected from an Affibody, a DARPin, an Anticalin, an Avimer, a Versabody or a Duocali; (b) a drug; and (c) a functional linker comprising a functional moiety which links the drug to the antibody mimetic, wherein when the conjugate is administered to a subject, compared to a conjugate without the functional moiety, the functional linker is able to: (i) increase tumor uptake, accumulation, and/or retention of the drug, (ii) decrease kidney uptake, accumulation, and/or retention of the drug, (iii) increase tumor/kidney ratio of the drug, (iv) prolong blood circulation of the drug and/or (v) extend half-life of the drug.

In some embodiments, a binding affinity of the conjugate to the tumor antigen is between about 10⁻¹² and about 10⁻⁸ M. In some embodiments, the functional moiety comprises one or more components selected from the group consisting of a polyethylene glycol (PEG) group, a renal enzyme cleavable group, a blood protein binding group, a spacer, and any combination thereof.

In some embodiments, the PEG group comprises a structure of

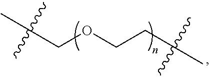

and wherein n is any integer in a range from about 1 to about 25. In some embodiments, the functional moiety comprises the blood protein binding group.

In some embodiments, the blood protein is selected from the group consisting of albumin, fetuin, transferrin, and IgG.

In some embodiments, the blood protein binding group comprises an albumin binding group. In some embodiments, the albumin binding group comprises a structure of

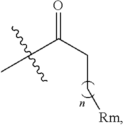

wherein n is any integer ranging from 1 to 20, Rₘ is H, CH₃, or COOH. In some embodiments, the albumin binding group comprises a structure of

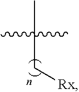

wherein Rₓ is selected from the group consisting of:

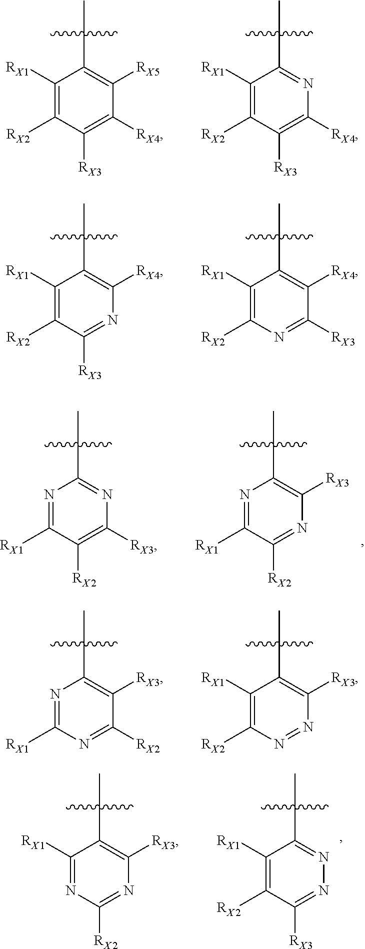

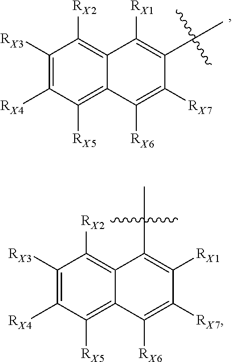

and wherein n is any integer ranging from 1 to 6, each of Rₓ₁, Rₓ₂, Rₓ₃, Rₓ₄, Rₓ₅, Rₓ₆, and Rₓ₇ is independently selected from the group consisting of H, N, S, O, Se, P, halogen, a C₅₋₂₀ aryl group, a C₁₋₂₀ alkyl group, a C₂₋₂₀ alkenyl group, and a C₂₋₂₀ alkynyl group, and wherein: (1) the C₅₋₂₀ aryl group, C₁₋₂₀ alkyl group, C₂₋₂₀ alkenyl group or the C₂₋₂₀ alkynyl group is unsubstituted or substituted by one or more substituents selected from the group consisting of N, S, O, Se, P, and halogen atom, (2) 0, 1 or 2 carbon atoms of the C₅₋₂₀ aryl group, C₁₋₂₀ alkyl group, C₂₋₂₀ alkenyl group or the C₂₋₂₀ alkynyl group are replaced by groups selected from the group consisting of C₆₋₁₀ arylene, 5 to 10 membered heteroarylene group, C₃₋₇ carbocyclylen, 5 to 10 membered heterocyclylene group, and wherein the arylene, heteroarylene, carbocyclylene and heterocyclylene groups are unsubstituted or substituted by one or more substituents selected from the group consisting of N, S, O, Se, P, halogen, C₁₋₆ alkyl, C₁₋₆ alkoxy, C₁₋₆ alkylthiol, —N(C₁₋₆ alkyl) (C₁₋₆ alkyl), nitro and sulfonic acid groups, and/or (3) 0, 1 or 2-CH— or —CH₂-groups of the C₅₋₂₀ aryl group, C₁₋₂₀ alkyl group, C₂₋₂₀ alkenyl group or the C₂₋₂₀ alkynyl group are replaced by groups selected from the group consisting of —O—, —S—, —S—S—, —C(O)— and —N(C₁₋₆ alkyl)-group. In some embodiments, the albumin binding group comprises a structure of

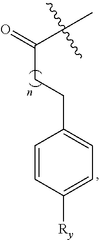

wherein R_{y} is selected from the group consisting of H, F, Cl, Br, I, —CH₃, —OCH₃, COOH, —CF₃, wherein n is any integer ranging from 1 to 6.

In some embodiments, the functional moiety comprises the renal enzyme cleavable group. In some embodiments, the renal enzyme cleavable group is selected from the group consisting of a brush border enzyme cleavable group, a lysosome cleavable enzyme cleavable group, and a combination thereof. In some embodiments, the renal enzyme cleavable group comprises a dipeptide or an oligopeptide. In some embodiments, the dipeptide or the oligopeptide comprises a structure selected from the group consisting of methionine-isoleucine, glycine-lysine, glycine-phenylalanine-lysine, methionine-valine-lysine, glycine-tyrosine, glycine-lysine-lysine, glycine-arginine-lysine, aspartic acid-glycine-lysine, methionine-glycine-lysine, methionine-isoleucine-lysine, glycine-tyrosine-lysine, glycine-valine, glycine-isoleucine, methionine-phenylalanine-lysine, glycine-(3-(2-naphthyl) alanine)-lysine, glycine-diphenylalanine-lysine, methionine-glycine-lysine, and any derivative thereof.

In some embodiments, the functional linker comprises a spacer. In some embodiments, the spacer comprises a structure selected from the group consisting of a natural amino acid residue, a non-natural amino acid residue, a C₅₋₂₀ aryl group, a C₁₋₂₀ alkyl group, a C₂₋₂₀ alkenyl group and a C₂₋₂₀ alkynyl group, and wherein: (i) the C₅₋₂₀ aryl group, C₁₋₂₀ alkyl group, C₂₋₂₀ alkenyl group or the C₂₋₂₀ alkynyl group is unsubstituted or substituted by one or more substituents selected from the group consisting of N, S, O, Se, P, and halogen atom, (ii) 0, 1 or 2 carbon atoms of the C₅₋₂₀ aryl group, C₁₋₂₀ alkyl group, C₂₋₂₀ alkenyl group or the C₂₋₂₀ alkynyl group are replaced by groups selected from the group consisting of C₆₋₁₀ arylene, 5 to 10 membered heteroarylene group, C₃₋₇ carbocyclylen, 5 to 10 membered heterocyclylene group, and wherein the arylene, heteroarylene, carbocyclylene and heterocyclylene groups are unsubstituted or substituted by one or more substituents selected from the group consisting of N, S, O, Se, P, halogen, C₁₋₆ alkyl, C₁-6 alkoxy, C₁₋₆ alkylthiol, —N(C₁₋₆ alkyl) (C₁₋₆ alkyl), nitro and sulfonic acid groups, and/or (iii) 0, 1 or 2-CH— or —CH₂-groups of the C₁₋₂₀ alkyl group, C₂₋₂₀ alkenyl group or the C₂₋₂₀ alkynyl group are replaced by groups selected from the group consisting of —O—, —S—, —S—S—, —C(O)— and —N(C₁₋₆ alkyl)-groups. In some embodiments, the amino acid residue is selected from the group consisting of lysine, aspartate, asparagine, diaminobutyric acid, phenylalanine, tyrosine, threonine, serine, proline, leucine, isoleucine, valine, arginine, histidine, glutamate, glutamine, and alanine.

In some embodiments, the functional linker further comprises a chelator group. In some embodiments, the chelator group comprises a structure selected from the group consisting of NODASA, NODAGA, TETA, TRITA, TRAP, DTPA, CHX-DTPA EDTA, CDTA, CPTA, DOTP, DOTPI, EGTA, HBED, TTHA, DTPA, DOTA, DOTAGA, NOTA, HP-DOA3, CBTE2a, TE2A, TMT, DPDP, HYNIC, DFO, HEDTA, NOPO, MAG3, NCS-MP-NODA, NH2-MPAA-NODA, H2DEDPA, H4octapa, Macropa, Pypa, Py4pa, THP, and SarAr.

In some embodiments, the functional linker is conjugated to the sdAb or the antibody mimetic via an amino acid of the sdAb or antibody mimetic. In some embodiments, the amino acid of the sdAb or antibody mimetic for conjugation is selected from the group consisting of cysteine, lysine, histidine, aspartate, glutamate, glutamine, arginine, tyrosine, tryptophan, threonine, and serine. In some embodiments, the functional linker comprises a conjugation group capable of conjugating with the sdAb or antibody mimetic. In some embodiments, the conjugation group is selected from the group consisting of an amine, a thiol, an alcohol, a ketone, an aldehyde, a nitrile, a carboxylic acid, an ester, an alkene, an alkyne, an anhydride, a succinimide, a maleimide, a phosphine, a disulfide, an alkoxyamine, an azide, an alkyl halide, an isothiocyanate (NCS), an epoxide, an isocyanate, a hydrazine, and an acyl halide.

In some embodiments, the tumor antigen is selected from the group consisting of prostate specific membrane antigen (PSMA), fibroblast activation protein alpha (FAP-alpha, FAP), a folate receptor, luteinizing hormone-releasing hormone (LHRH), noradrenaline transporter (NAT), epidermal growth factor receptor (EGFR), Human Epidermal Growth Factor Receptor-2 (HER-2), vascular endothelial growth factor (VGFR), Mucin-1 (MUC-1), Mucin-4 (MUC-4), urokinase-type plasminogen activator receptor (uPAR), tumor-associated glycoprotein 72 (TAG-72), Claudin 18.2 (CLDN18.2), vascular endothelial growth factor receptor (VEGFR), C—X—C chemokine receptor type 4 (CXCR4), Hepsin, TMPRSS2, and cMET.

In some embodiments, the tumor antigen is CLDN18.2. In some embodiments, the sdAb comprises a CDR1, CDR2 and CDR3 as set forth in SEQ ID NO. 1. In some embodiments, the sdAb comprises: (1) a CDR1 comprising an amino acid sequence as set forth in SEQ ID NO. 2 and an amino acid sequence with one or more amino acid alterations as compared to SEQ ID NO. 2, (2) a CDR2 comprising an amino acid sequence as set forth in SEQ ID NO. 3 and an amino acid sequence with one or more amino acid alterations as compared to SEQ ID NO. 3, and (3) a CDR3 comprising an amino acid sequence as set forth in SEQ ID NO. 4 and an amino acid sequence with one or more amino acid alterations as compared to SEQ ID NO. 4.

In some embodiments, the tumor antigen is FAP. In some embodiments, the sdAb comprises a CDR1, CDR2 and CDR3 as set forth in SEQ ID NO. 5. In some embodiments, the sdAb comprises: (1) a CDR1 comprising an amino acid sequence as set forth in SEQ ID NO. 6 and an amino acid sequence with one or more amino acid alterations as compared to SEQ ID NO. 6, (2) a CDR2 comprising an amino acid sequence as set forth in SEQ ID NO. 7 and an amino acid sequence with one or more amino acid alterations as compared to SEQ ID NO. 7, and (3) a CDR3 comprising an amino acid sequence as set forth in SEQ ID NO. 8 and an amino acid sequence with one or more amino acid alterations as compared to SEQ ID NO. 8.

In some embodiments, the drug is selected from the group consisting of a chemotherapeutic agent, a toxin, a cytokine, an enzyme, an immunomodulator, a chelating complex, a diagnostic agent, a nanoparticle, and a radioisotope. In some embodiments, the drug is a radioisotope selected from the group consisting of bismuth-213, caesium-131, caesium-137, c caesium-131, cobalt-60, holmium-166, iodine-125, iodine-131, iridium-192, lead-212, lutetium-177, palladium-103, phosphorus-32, potassium-42, radium-223, rhenium-186, rhenium-188, samarium-153, scandium-47, selenium-75, sodium-24, strontium-89, technetium-99m, thorium-227, xenon-133, ytterbium-169, ytterbium-177, yttrium-90, actinium-225, astatine-211, bismuth-213, carbon-11, nitrogen-13, oxygen-15, fluorine-18, cobalt-57, copper-64, copper-67, gallium-67, gallium-68, indium-111, iodine-123, iodine-124, krypton-81m, rubidium-82, strontium-82, thallium-201, and zirconium-89.

In another aspect, provided is a method for treating a disorder in a subject in need thereof, comprising administering an effective amount of the conjugate of the present application to the subject. In some embodiments, the disorder is cancer.

In another aspect, provided is a method of preparing the conjugate of the present application, comprising conjugating the functional linker and the drug to the sdAb.

### BRIEF DESCRIPTION OF THE DRAWINGS

FIG. 1 depicts an exemplary synthesis scheme of the functional linker of the present application.

FIG. 2 depicts serum concentration of the CLDN18.2 sdAb-linker conjugates over 48 hours.

FIG. 3 depicts serum concentration of the FAP sdAb-linker conjugates over 48 hours.

FIG. 4 depicts in vivo SPECT imaging after injection of the CLDN18.2-linker conjugates over 48 hours.

FIG. 5 depicts in vivo SPECT imaging after injection of the FAP-linker conjugates over 168 hours.

### DETAILED DESCRIPTION

### Definition

Unless specifically indicated otherwise, all technical and scientific terms used herein have the same meaning as commonly understood by those of ordinary skill in the art to which this application belongs. In addition, any method or material similar or equivalent to a method or material described herein can be used in the practice of the present application. For purposes of the present application, the following terms are defined.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X.”

The term “about X-Y” used herein has the same meaning as “about X to about Y.” The expression “about X, Y and/or Z” used herein has the same meaning as “about X, about Y, and/or about Z.”

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “effective amount” used herein refers to an amount of a compound, a composition, or a conjugate sufficient to treat a specified disorder, condition, or disease, such as to ameliorate, palliate, lessen, and/or delay one or more of its symptoms in a subject.

As is understood in the art, an “effective amount” may be in one or more doses, e.g., a single dose or multiple doses may be required to achieve the desired treatment endpoint.

As used herein, “therapeutically effective amount” indicates an amount administered to the subject that results in a desired pharmacological and/or physiological effect for the condition in the subject. The effect in the subject may be prophylactic in terms of completely or partially preventing a condition or symptom thereof and/or may be therapeutic in terms of a partial or complete cure for the condition and/or adverse effect attributable to the condition.

As used herein, the term “subject” refers to an organism to be treated by the methods of the present invention. Such organisms are preferably mammals (e.g., murines, simians, equines, bovines, porcines, canines, felines, and the like), and more preferably humans.

As described herein, “tumor antigen” refers to an antigenic substance produced at a tumor site.

As described herein, “single-photon emission computed tomography” (or SPECT) is a nuclear imaging modality used in diagnostic medicine. SPECT produces a 3-dimensional image of the distribution of a radioactive tracer (or probe) injected into the bloodstream and subsequently taken up by certain tissues. This is accomplished via the use of specialized nuclear medicine cameras. Thus, SPECT allows for an assessment of the perfusion and functionality of specific tissues.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity.

A full-length antibody comprises two heavy chains and two light chains. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (al heavy chain), or IgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (dsdiabody), a single-chain Fv (scFv), an scFv dimer (bivalent diabody), a multispecific antibody formed from a portion of an antibody comprising one or more CDRs, a single domain antibody (e.g., a camelized single domain antibody), a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “single-domain antibody (sdAb, also known as nanobody)” as used herein refers to an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (~50 kDa, one light chain and half a heavy chain) and single-chain variable fragments (~25 kDa, two variable domains, one from a light and one from a heavy chain).

The first single-domain antibodies were engineered from heavy-chain antibodies found in camelids; these are called VHH fragments. Cartilaginous fishes also have heavy-chain antibodies (IgNAR, immunoglobulin new antigen receptor), from which single-domain antibodies called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes.

Camelid nanobodies have been shown to be as specific as antibodies, and in some cases they are more robust. They are easily isolated using the same phage panning procedure used for antibodies, allowing them to be cultured in vitro in large concentrations. The smaller size and single domain make these antibodies easier to transform into bacterial cells for bulk production, making them ideal for research purposes.

As used herein, the term “CDR” or “complementarity determining region” is intended to mean the non-contiguous antigen combining sites found within the variable region of both heavy and light chain polypeptides. These particular regions have been described by Kabat, et al. (1977) J. Biol. Chem. 252:6609-6616; Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45:3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR Definitions

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

In some embodiments, the CDRs of an antibody can be determined according to the IMGT numbering system. With respect to the IMGT numbering system, (i) the VH CDR1 is typically present at amino acid positions 25 to 35 of the heavy chain; (ii) the VH CDR2 is typically present at amino acid positions 51 to 57 of the heavy chain; and (iii) the VH CDR2 is typically present at amino acid positions 93 to 102 of the heavy chain. With respect to the IMGT numbering system, (i) the VL CDR1 is typically present at amino acid positions 27 to 32 of the light chain; (ii) the VL CDR2 is typically present at amino acid positions 50 to 52 of the light chain; and (iii) the VL CDR3 is typically present at amino acid positions 89 to 97 of the light chain.

“Framework” or “FR” residues are those variable-domain residues other than the CDR residues as herein defined.

“Percent (%) amino acid sequence identity” or “homology” with respect to the polypeptide and antibody sequences identified herein is defined as the percentage of amino acid residues in a candidate sequence that are identical with the amino acid residues in the polypeptide or antibody being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32 (5): 1792-1797, 2004; Edgar, R. C., BMC Bioinformatics 5 (1): 113, 2004).

As used herein, the term “antibody mimetic” refers to molecules capable of mimicking an antibody's ability to bind an antigen, but which are not limited to native antibody structures. Examples of such antibody mimetics include, but are not limited to, Affibodies, DARPins, Anticalins, Avimers, and Versabodies.

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55.). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains. Additional information regarding DARPins and other DRP technologies can be found in US Patent Application Publication No. 2004/0132028, and International Patent Application Publication No. WO 02/20565, both of which are hereby incorporated by reference in their entirety.

Anticalins are an additional antibody mimetic technology, however in this case the binding specificity is derived from lipocalins, a family of low molecular weight proteins that are naturally and abundantly expressed in human tissues and body fluids. Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains. Additional information regarding Anticalins can be found in U.S. Pat. No. 7,250,297 and International Patent Application Publication No. WO 99/16873, both of which are hereby incorporated by reference in their entirety.

Another antibody mimetic technology useful in the present application is Avimer. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in E. coli, improved thermostability and resistance to proteases. Avimers with sub-nanomolar affinities have been obtained against a variety of targets. Additional information regarding Avimers can be found in US Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabody is another antibody mimetic technology that could be used in the present application. Versabodies are small proteins of 3-5 kDa with >15% cysteines, which form a high disulfide density scaffold, replacing the hydrophobic core that typical proteins have. Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life. Additional information regarding Versabodies can be found in US Patent Application Publication No. 2007/0191272 which is hereby incorporated by reference in its entirety.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, the terms “specifically binds,” “specifically recognizing,” and “is specific for” refer to measurable and reproducible interactions, such as binding between an antibody and an antigen thereof, which is determinative of the presence of the target or antigen in the presence of a heterogeneous population of molecules, including biological molecules. For example, an antibody that specifically recognizes an antigen is the antibody that binds this antigen with greater affinity, avidity, more readily, and/or with greater duration than its bindings to other targets or antigens. In some embodiments, the extent of binding of antibody to an unrelated target or antigen is less than about 10% of the binding of the antibody to the antigen thereof as measured, e.g., by a radioimmunoassay (RIA). In some embodiments, an antibody that specifically binds the antigen thereof has a dissociation constant (K_{D}) of ≤10⁻⁸ M, ≤10⁻⁹ M, ≤10⁻¹⁰ M, ≤10⁻¹¹ M, or ≤10⁻¹² M. In some embodiments, said specific binding can include, but does not require exclusive binding. Binding specificity of the antibody can be determined experimentally by methods known in the art. Such methods comprise, but are not limited to, e.g., Western blots, ELISA-, RIA-, ECL-, IRMA-, EIA-, BIACORE™-tests and peptide scans.

As used herein, the term “conjugate” refers to a molecule which comprises more than one functional moieties, in which a first functional moiety and a second functional moiety are linked (e.g. covalently linked) to one another via a linker, a spacer or a reactive linking group, as described herein.

As used herein, the term “PEG (polyethylene glycol or polyethylene glycol)” refers to a water-soluble poly(ethylene oxide) with the structure “—(OCH2CH2)ₙ-.” In some embodiments, n is in a range from about 1 to about 25. As used herein, PEG also includes “—CH2CH2-O(CH2CH2O)ₙ—CH2CH2-” and “—(OCH2CH2)ₙO—,” depending upon whether or not the terminal oxygens have been displaced. Throughout the present application, it should be understood that the term “PEG” includes structures having various terminal groups and so forth. The term “PEG” also means a polymer that contains a majority, that is to say, greater than 50%, of —OCH₂CH₂-repeating subunits. With respect to specific forms, the PEG encompasses those branched, linear, and forked structures.

As used herein, groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth.

As used herein, the term “aromatic” or “aryl,” refers to a closed ring structure which has at least one ring having a conjugated pi electron system and includes both carbocyclic aryl and heterocyclic aryl (or “heteroaryl” or “heteroaromatic”) groups. The carbocyclic or heterocyclic aromatic group may contain from 5 to 20 ring atoms. The term includes monocyclic rings linked covalently or fused-ring polycyclic (i.e., rings which share adjacent pairs of carbon atoms) groups. An aromatic group can be unsubstituted or substituted. Non-limiting examples of “aromatic” or “aryl” groups include phenyl, 1-naphthyl, 2-naphthyl, 4-biphenyl, anthracenyl, and phenanthracenyl. Substituents for each of the above aryl and heteroaryl ring systems are selected from the group of acceptable substituents described herein. The term “arylene” can be construed accordingly.

As used herein, the term “alkyl” includes both saturated linear chain and branched alkyl groups. In some embodiments, an alkyl group includes but is not limited to a C₁₋₂₀ alkyl group, a C₁₋₁₅, a C₁₋₁₂ alkyl group, a C₁-6 alkyl group and a C₁₋₄ alkyl group. In some embodiments, the alkyl groups include, for example, methyl, ethyl, propyl, isopropyl, butyl, isobutyl, tert-butyl, pentyl and hexyl. The term “alkylene” can be construed accordingly.

As used herein, the term “alkenyl” refers to a group containing one or more carbon-carbon double bonds, which may be linear or branched. In some embodiments, the alkenyl group includes but is not limited to a C₂₋₂₀ alkenyl group, a C₂₋₁₅ alkenyl group, a C₂₋₁₂ alkenyl group, a C₂₋₆ alkenyl group, and a C₂₋₄ alkenyl group. The term “alkenylene” can be construed accordingly.

As used herein, the term “alkynyl” refers to a carbon chain containing one or more triple bonds, which may be linear or branched. In some embodiments, the alkynyl group includes but is not limited to a C₂₋₂₀ alkynyl group, a C₂₋₁₅ alkynyl group, a C₂₋₁₂ alkynyl group, a C₂₋₆ alkynyl group and a C₂₋₄ alkynyl group. The term “alkynylene” can be construed accordingly.

In some embodiments, the alkyl, alkenyl or alkynyl group is unsubstituted. In some embodiments, alkyl, alkenyl or alkynyl group is substituted. In some embodiments, a substituted alkyl, alkenyl or alkynyl group has from 1 to 10 substituents, 1 to 5 substituents, or 1, 2 or 3 substituents. In some embodiments, a substituted alkyl, alkenyl or alkynyl group carries a sulfonic acid substituent and/or a Halogen substituent.

In some embodiments, an alkyl, alkenyl, alkynyl group or an arylene, alkylene, alkenylene, alkynylene group comprises (a) 0, 1 or 2 carbon atoms may be substituted by groups selected from the group consisting of C₆₋₁₀ arylene, 5 to 10 membered heteroarylene, C₃₋₇ carbocyclylene and 5 to 10 membered heterocyclylene groups, and (b) 0, 1 or 2-CH₂-groups may be replaced by groups selected from the group consisting of —O—, —S—, —S—S—, —C(O)— and —N(C₁₋₆ alkyl)-groups.

As used herein, a C₆₋₁₀ aryl group is a monocyclic or polycyclic 6 to 10 membered aromatic hydrocarbon ring system having from 6 to 10 carbon atoms, such as Phenyl. As used herein, a 5 to 10 membered heteroaryl group is a monocyclic or polycyclic 5 to 10 membered aromatic ring system, such as a 5 or 6 membered ring, containing at least one heteroatom, for example 1, 2, 3 or 4 heteroatoms, such as O, S and N. The term “heteroarylene” should be construed accordingly.

Examples of monocyclic heteroaryl groups include but are not limited to thienyl, furyl, pyrrolyl, imidazolyl, thiazolyl, isothiazolyl, pyrazolyl, oxazolyl, isoxazolyl, triazolyl, thiadiazolyl, oxadiazolyl, pyridinyl, pyridazinyl, pyrimidinyl, pyrazinyl, triazinyl and tetrazolyl groups. Examples of polycyclic heteroaryl groups include but are not limited to benzothienyl, benzofuryl, benzimidazolyl, benzothiazolyl, benzisothiazolyl, benzoxazolyl, benzisoxazolyl, benztriazolyl, indolyl, isoindolyl and indazolyl groups. Preferred polycyclic groups include indolyl, isoindolyl, benzimidazolyl, indazolyl, benzofuryl, benzothienyl, benzoxazolyl, benzisoxazolyl, benzothiazolyl and benzisothiazolyl groups, more preferably benzimidazolyl, benzoxazolyl and benzothiazolyl, most preferably benzothiazolyl. However, monocyclic heteroaryl groups are preferred. In some embodiments, the heteroaryl group is a 5- to 6-membered heteroaryl group. Particularly preferred heteroaryl groups are thienyl, pyrrolyl, imidazolyl, thiazolyl, isothiazolyl, pyrazolyl, oxazolyl, isoxazolyl, triazolyl, pyridinyl, pyridazinyl, pyrimidinyl and pyrazinyl groups. In some embodiments, the heteroaryl group includes but is not limited to thienyl, pyridinyl, pyridazinyl, pyrimidinyl, pyrazinyl, pyrrolyl and triazinyl, most preferably pyridinyl.

As used herein, a 5 to 10 membered heterocyclyl group is a non-aromatic, saturated or unsaturated, monocyclic or polycyclic C₅₋₁₀ carbocyclic ring system in which one or more, for example 1, 2, 3 or 4, of the carbon atoms are replaced with a moiety selected from the group consisting of N, O, S, S(O) and S(O)₂. In some embodiments, the 5 to 10 membered heterocyclyl group is a 5 to 6 membered ring. The term “heterocyclyene” should be construed accordingly.

Examples of heterocyclyl groups include azetidinyl, oxetanyl, thietanyl, pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, methylenedioxyphenyl, ethylenedioxyphenyl, thiomorpholinyl, S-oxo-thiomorpholinyl, S,S-dioxo-thiomorpholinyl, morpholinyl, 1,3-dioxolanyl, 1,4-dioxolanyl, trioxolanyl, trithianyl, imidazolinyl, pyranyl, pyrazolinyl, thioxolanyl, thioxothiazolidinyl, 1H-pyrazol-5-(4H)-onyl, 1,3,4-thiadiazol-2 (3H)-thionyl, oxopyrrolidinyl, oxothiazolidinyl, oxopyrazolidinyl, succinimido and maleimido groups and moieties. Preferred heterocyclyl groups are pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, thiomorpholinyl and morpholinyl groups and moieties. More preferred heterocyclyl groups are tetrahydropyranyl, tetrahydrothiopyranyl, thiomorpholinyl, tetrahydrofuranyl, tetrahydrothienyl, piperidinyl, morpholinyl and pyrrolidinyl groups.

As used herein, a C₃₋₇ carbocyclyl group is a non-aromatic saturated or unsaturated hydrocarbon ring having from 3 to 7 carbon atoms. In some embodiments, it is a saturated or mono-unsaturated hydrocarbon ring (i.e. a cycloalkyl moiety or a cycloalkenyl moiety) having from 3 to 7 carbon atoms, more preferably having from 5 to 6 carbon atoms. Examples include cyclopropyl, cyclobutyl, cyclopentyl and cyclohexyl and their mono-unsaturated variants. Particularly preferred carbocyclic groups are cyclopentyl and cyclohexyl. The term “carbocyclylene” should be construed accordingly.

As used herein, the term “halogen” or “halide” can be used interchangeably and includes but is not limited to F, Cl, Br and I.

As used herein, the term “reactive linking group” is a reactive group capable of joining two or more chemical groups, chemical moieties or units by a covalent bond. The reactive linking group may be used to attach one component selected from the group consisting of PEG group, a renal enzyme cleavable group, a blood protein binding group, a spacer, and chelator to another. In some embodiments, the reactive linking group includes but is not limited to amine, imine, carboxylate, alcohol, thiol, selenol, phenol, ester, acetone, aldehyde, carbene, sulfonyl halide, imidate, anhydride, disulfide, maleimide, phosphine, disulfide, alkoxyamine, azide, alkyne, strained alkyne, strained alkene, halogen, sulfonate, haloacetyl, hydrazide, diazirine, phosphine, tetrazine, isothiocyanate, and oxaziridine.

As used herein, the term “protecting group” refers to a moiety, that when attached to a reactive group in a molecule masks, reduces or prevents that reactivity. Examples of protecting groups can be found in T. W. Greene and P. G M. Wuts, Protective Groups in Organic Synthesis, 3rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include but are not limited to acyl groups, benzyl and trityl ethers, tetrahydropyranyl ethers, trialkylsilyl ethers and allyl ethers. Representative amino protecting groups include but are not limited to formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

As used herein, the term “chelator” or “chelator group” refers to a moiety that is capable of coordinating (or binding) a metal ion in a polydentate (e.g., coordination via two or more atoms of moieties) fashion. In some embodiments, the chelator may include donor atoms that bind to the metal, such as S, N, or O. For example, the chelator may be a tridentate ligand that has three donor atoms. In some embodiments, the chelator group is capable of coordinating a radioisotope as described herein.

As used herein, the term “pharmacokinetic profile” or “PK profile” refers to a profile of drug concentration in blood or plasma. Such a profile can be a relationship of drug concentration over time (i.e., a “concentration-time PK profile”) or a relationship of drug concentration versus number of doses ingested (i.e., a “concentration-dose PK profile”). A PK profile is characterized by PK parameters.

As used herein the term “pharmacokinetic parameter” or “PK parameter” refers to a measure of drug concentration in blood or plasma, such as: (1) “drug Cmax,” the maximum concentration of drug achieved in blood or plasma; (2) “drug Tmax,” the time elapsed following ingestion to achieve Cmax; and (3) “drug exposure,” the total concentration of drug present in blood or plasma over a selected period of time, which can be measured using the area under the curve (AUC) of a time course of drug release over a selected period of time (t). Modification of one or more PK parameters provides for a modified PK profile.

As used herein, the term “pharmacodynamic (PD) profile” or “PD profile” refers to a profile of the efficacy of a drug in a patient (or subject), which is characterized by PD parameters.

As used herein, the term “PK parameter” or “PD parameter” include “drug Emax” (the maximum drug efficacy), “drug EC₅₀” (the concentration of drug at 50% of the Emax) and side effects.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The disclosures of all publications, patents, patent applications and published patent applications referred to herein are hereby incorporated herein by reference in their entirety.

### Conjugates

In one aspect, the present invention provides a conjugate comprising: (1) an sdAb that specifically binds to the tumor antigen, (2) a drug, and (3) a functional linker comprising a functional moiety which links the drug to the sdAb.

In some embodiments of the conjugate, when the conjugate is administered to a subject, as compared to a conjugate without the functional moiety, the functional linker can: (i) modify PK profile of the conjugate, (ii) modify PD profile of the conjugate, (iii) modify biodistribution of the conjugate, (iv) improve efficacy of the conjugate, (v) result in a decrease in the nephrotoxicity and increase in therapeutic efficacy in a subject. In some embodiments, when the conjugate is administered to a subject, as compared to a conjugate without the functional moiety, the functional linker can achieve at least one of (i)-(v) as described above. In some embodiments, when the conjugate is administered to a subject, as compared to a conjugate without the functional moiety, the functional linker can achieve at least two of (i)-(v) as described above. In some embodiments, when the conjugate is administered to a subject, as compared to a conjugate without the functional moiety, the functional linker can achieve at least three of (i)-(v) as described above. In some embodiments, when the conjugate is administered to a subject, as compared to a conjugate without the functional moiety, the functional linker can achieve at least four of (i)-(v) as described above. In some embodiments, when the conjugate is administered to a subject, as compared to a conjugate without the functional moiety, the functional linker can achieve all of (i)-(v) as described above.

In another aspect, the present application provides a conjugate comprising: (a) a single-domain antibody (sdAb) that specifically binds to a tumor antigen; (b) a drug; and (c) a functional linker comprising the functional moiety as described herein, wherein the functional linker increases tumor uptake, accumulation, and/or retention of the drug when the conjugate is administered to a subject compared to a conjugate without the functional moiety.

In another aspect, provided is a conjugate comprising: (a) a single-domain antibody (sdAb) that specifically binds to a tumor antigen; (b) a drug; and (c) a functional linker comprising the functional moiety as described herein, wherein the functional linker decreases kidney uptake, accumulation, and/or retention of the drug when the conjugate is administered to a subject compared to a conjugate without the functional moiety.

In another aspect, provided is a conjugate comprising: (a) a single-domain antibody (sdAb) that specifically binds to a tumor antigen; (b) a drug; and (c) a functional linker comprising the functional moiety as described herein, wherein the functional linker increases tumor/kidney ratio of the drug when the conjugate is administered to a subject compared to a conjugate without the functional linker.

In another aspect, provided is a conjugate comprising: (a) a single-domain antibody (sdAb) that specifically binds to a tumor antigen; (b) a drug; and (c) a functional linker as described herein, wherein the functional linker prolongs blood circulation of the drug when the conjugate is administered to a subject compared to a conjugate without the functional linker.

In another aspect, provided is a conjugate comprising: (a) a single-domain antibody (sdAb) that specifically binds to a tumor antigen; (b) a drug; and (c) a functional linker as described herein, wherein the functional linker extends half-life of the drug when the conjugate is administered to a subject compared to a conjugate without the functional linker.

In another aspect, provided is a conjugate comprising: (a) a single-domain antibody (sdAb) that specifically binds to a tumor antigen; (b) a drug; and (c) a functional linker as described herein, wherein the functional linker comprises at least one functional moiety (such as at least two or at least three) selected from the group consisting of a PEG group, a renal enzyme cleavable group, a spacer and a blood protein binding group.

In some embodiments, the functional linker in the conjugate is capable of achieving at least one, at least two, at least three, at least four or all of the following functions: (i) increasing tumor uptake, accumulation, and/or retention of the drug when the conjugate is administered to a subject compared to a conjugate without the functional linker; (ii) decreasing kidney uptake, accumulation, and/or retention of the drug when the conjugate is administered to a subject compared to a conjugate without the functional linker; (iii) increasing tumor/kidney ratio of the drug when the conjugate is administered to a subject compared to a conjugate without the functional linker; (iv) prolonging blood circulation of the drug when the conjugate is administered to a subject compared to a conjugate without the functional linker; and (v) extending half-life of the drug, when the conjugate is administered to a subject compared to a conjugate without the functional linker.

In another aspect, provided herein is conjugate comprising: (a) an antibody mimetic that specifically binds to a tumor antigen; (b) a drug; and (c) a functional linker comprising the functional moiety as described herein. In some embodiments, the antibody mimetic is selected from the group consisting of an Affibody, a DARPin, an Anticalin, an Avimer, a Versabody and a Duocali. In some embodiments, the molecular weight of the conjugate is no more than about 60 KDa, such as no more than about 60, about 55, about 50, about 45, about 40, about 35, about 30, about 25, about 20, about 15, about 10, about 5, or about 3 KDa.

### Functional Linker

In another aspect, provided herein is a functional linker used in the conjugate as described herein, wherein the functional linker comprises a functional moiety. In some embodiments, the functional linker is capable of achieving at least one of the functions including: (i) increasing tumor uptake, accumulation, and/or retention of the conjugate when the conjugate is administered to a subject compared to a conjugate without the functional linker; (ii) decreasing kidney uptake, accumulation, and/or retention of the conjugate when the conjugate is administered to a subject compared to a conjugate without the functional linker; (iii) increasing tumor/kidney ratio of the conjugate when the conjugate is administered to a subject compared to a conjugate without the functional linker; (iv) prolonging blood circulation of the conjugate when the conjugate is administered to a subject compared to a conjugate without the functional linker; and (v) extending half-life of the conjugate when the conjugate is administered to a subject compared to a conjugate without the functional moiety.

In some embodiments, the functional linker of the present application comprises one or more components selected from the group consisting of a polyethylene glycol (PEG) group, a renal enzyme cleavable group, a blood protein binding group, a spacer, and any derivative thereof.

In some embodiments where the functional linker comprises the PEG group, the PEG group can increase the hydrophilicity of the conjugate for better clearance and/or is useful as a spacer for separating different functional moieties. The PEG group may also prolong circulation time and reduce kidney uptake, accumulation and/or retention of the drug. In some embodiments, the functional linker comprises one PEG group. In some embodiments, the functional linker comprises more than one PEG groups. In some embodiments, the functional linker comprises 2, 3, 4, or 5 PEG groups as described herein.

In some embodiments, the PEG group comprises about 1 to about 25 monomeric units. In some embodiments, the PEG group comprises about 1 to about 5 monomeric units. In some embodiments, the PEG group comprises about 5 to about 10 monomeric units. In some embodiments, the PEG group comprises about 10 to about 15 monomeric units. In some embodiments, the PEG group comprises about 15 to about 20 monomeric units. In some embodiments, the PEG group comprises about 20 to about 25 monomeric units. In some embodiments, the PEG group comprises 1, 2, 3, 4, 5, 6, 7, 8, 9, 10, 11, 12, 13, 14, 15, 16, 17, 18, 19, 20, 21, 22, 23, 24 or 25 monomeric units.

In some embodiments, the PEG group comprises a structure of

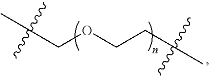

where n is any integer in a range from about 1 to about 25. In some embodiments, the PEG group comprises a structure of

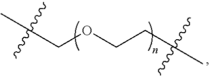

where n is any integer in a range from about 1 to about 5.

In some embodiments, the PEG group comprises a structure of

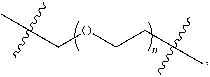

where n is any integer in a range from about 5 to about 10. In some embodiments, the PEG group comprises a structure of

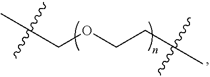

where n is any integer in a range from about 10 to about 15. In some embodiments, the PEG group comprises a structure of

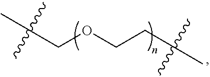

where n is any integer in a range from about 15 to about 20. In some embodiments, the PEG group comprises a structure of

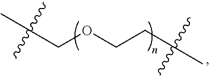

where n is any integer in a range from about 20 to about 25. In some embodiments, the PEG group comprises a structure of

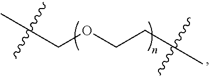

where n is 1, 2, 3, 4, 5, 6, 7, 8, 9, 10, 11, 12, 13, 14, 15, 16, 17, 18, 19, 20, 21, 22, 23, 24 or 25.

In some embodiments, the functional linker comprises a blood protein binding group. In some embodiments, the blood protein is selected from the group consisting of albumin, fetuin, transferrin, and IgG. In some embodiments, the blood protein binding group comprises an albumin binding group or an albumin binder.

Albumin is a protein produced by the liver that acts as a key lipid delivery vehicle for tissues. Short- to medium-length fatty acids (6 to 12 carbons) bind albumin with affinities between about 0.5 μM and about 60 μM, while longer fatty acids (14 to 18 carbons) have 10-fold higher affinities (e.g., below 50 nM). Any suitable albumin binding group known in the art can be used in the functional linker of the present application. In some embodiments where the functional linker comprises the albumin binding group, the binding affinity of the conjugate to human serum albumin is between about 10⁻⁷ and about 10⁻⁴ M. In some embodiments where the functional linker comprises the albumin binding group, the binding affinity of the conjugate to human serum albumin is in a range between about 10⁻⁶ M and about 10⁻⁴ M. In some embodiments where the functional linker comprises the albumin binding group, the binding affinity of the conjugate to human serum albumin is in a range between about 10⁻⁵ M and about 10⁻⁴ M. In some embodiments where the functional linker comprises the albumin binding group, the binding affinity of the conjugate to human serum albumin is in a range between about 10⁻⁷ M and about 10⁻⁵ M. In some embodiments where the functional linker comprises the albumin binding group, the binding affinity of the conjugate to human serum albumin is in a range between about 10⁻⁷M and about 10⁻⁶ M. In some embodiments where the functional linker comprises the albumin binding group, the binding affinity of the conjugate to human serum albumin is in a range between about 10⁻⁶ M and about 10⁻⁵ M.

Exemplary albumin binding group that can be used in the functional liker of the present application includes but is not limited to a short-chain fatty acid, a medium-chain chain fatty acid, a long-chain fatty acid, a myristic acid, a substituted or unsubstituted indole-2-carboxylic acid, a substituted or unsubstituted thioamide, a substituted or unsubstituted 4-oxo-4-(5,6,7,8-tetrahydronaphthalen-2-yl) butanoic acid, a substituted or unsubstituted naphthalene acylsulfonamide, a substituted or unsubstituted diphenylcyclohexanol phosphate ester, a substituted or unsubstituted 4-iodophenylalkanoic acid, a substituted or unsubstituted 3-(4-iodophenyl) propionic acid, a substituted or unsubstituted 2-(4-iodophenyl) acetic acid, or a substituted or unsubstituted 4-(4-iodophenyl) butanoic acid. In some embodiments, the blood protein binder group comprises a fatty acid, a butyric acid, or a derivative thereof. In some embodiments, blood protein binder group comprises comprise: 3-iodo-tyrosine, a short- or medium-chain fatty acids, a long-chain fatty acid, 4-(p-iodophenyl) butyric acid, or 4-(p-methyl) butyric acid.

In some embodiments, the albumin binding group that can be used in the functional liker of the present application comprises a structure of

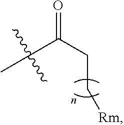

wherein n is any integer ranging from 1 to 20, Rₘ is H, CH₃ or COOH.

In some embodiments, the albumin binding group that can be used in the functional liker of the present application comprises a structure of

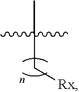

wherein Rₓ is selected from the group consisting of

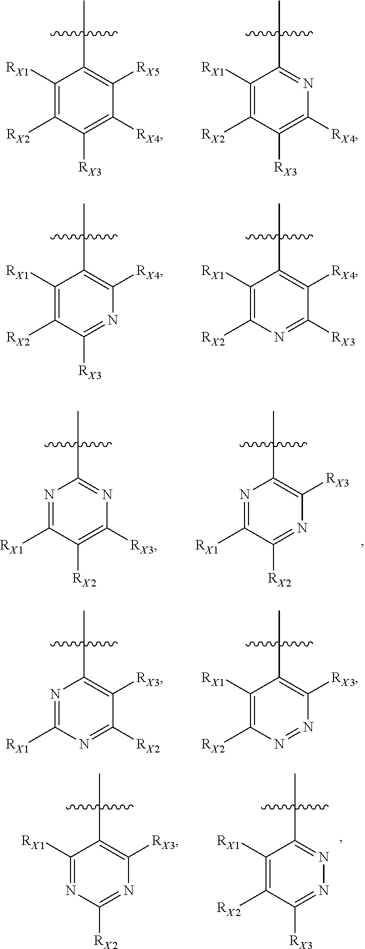

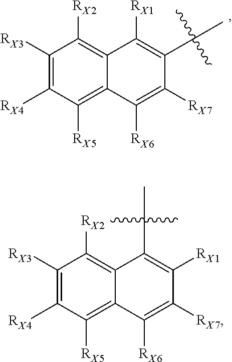

and
where n is any integer ranging from 1 to 6,
each of Rₓ₁, Rₓ₂, Rₓ₃, Rₓ₄, Rₓ₅, Rₓ₆, and Rₓ₇ is independently selected from the group consisting of H, N, S, O, Se, P, halogen, a C₅₋₂₀ aryl group, a C₁₋₂₀ alkyl group, a C₂₋₂₀ alkenyl group, and a C₂₋₂₀ alkynyl group. In some embodiments, the C₅₋₂₀ aryl group, C₁₋₂₀ alkyl group, C₂₋₂₀ alkenyl group or the C₂₋₂₀ alkynyl group can be unsubstituted or substituted by one or more substituents selected from the group consisting of N, S, O, Se, P, and halogen atom. In some embodiments, 0, 1 or 2 carbon atoms of the C₅₋₂₀ aryl group, C₁₋₂₀ alkyl group, C₂₋₂₀ alkenyl group or the C₂₋₂₀ alkynyl group are replaced by groups selected from the group consisting of C₆₋₁₀ arylene, 5 to 10 membered heteroarylene group, C₃₋₇ carbocyclylen, 5 to 10 membered heterocyclylene group, and wherein the arylene, heteroarylene, carbocyclylene and heterocyclylene groups are unsubstituted or substituted by one or more substituents selected from the group consisting of N, S, O, Se, P, halogen, C₁₋₆ alkyl, C₁₋₆ alkoxy, C₁₋₆ alkylthiol, —N(C₁₋₆ alkyl) (C₁₋₆ alkyl), nitro and sulfonic acid groups. In some embodiments, 0, 1 or 2-CH— or —CH₂-groups of the C₅₋₂₀ aryl group, C₁₋₂₀ alkyl group, C₂₋₂₀ alkenyl group or the C₂₋₂₀ alkynyl group are replaced by groups selected from the group consisting of —O—, —S—, —S—S—, —C(O)— and —N(C₁₋₆ alkyl)-group.

In some embodiments, the albumin binding group that can be used in the functional liker of the present application comprises a structure of

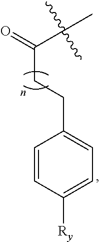

where R_{y} is selected from the group consisting of H, F, Cl, Br, I, —CH₃, —OCH₃, COOH, —CF₃, and n is any integer ranging from 1 to 6.

In some embodiments, the albumin binding group comprises a structure selected from the group consisting of:

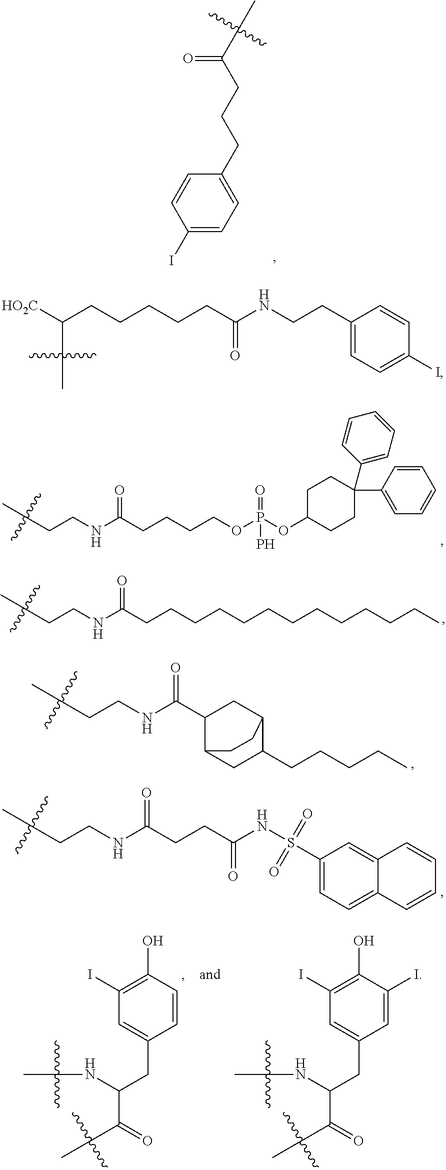

In some embodiments, the functional linker comprises the renal enzyme cleavable group which accelerates radiometabolite excretion into the urine of the subject. In some embodiments, the renal enzyme cleavable group is selected from the group consisting of a brush border enzyme cleavable group, a lysosome cleavable enzyme cleavable group, and a combination thereof.

In some embodiments, the renal enzyme cleavable group comprises an oligopeptide. In some embodiments, the oligopeptide comprises a dipeptide or derivative thereof. In some embodiments, the oligopeptide comprises a tripeptide or derivative thereof. In some embodiments, the oligopeptide comprises a structure selected from the group consisting of: methionine-isoleucine, glycine-lysine, glycine-phenylalanine-lysine, methionine-valine-lysine, glycine-tyrosine, glycine-lysine-lysine, glycine-arginine-lysine, aspartic acid-glycine-lysine, methionine-glycine-lysine, methionine-isoleucine-lysine, glycine-tyrosine-lysine, glycine-valine, glycine-isoleucine, methionine-phenylalanine-lysine, glycine-(3-(2-naphthyl) alanine)-lysine, glycine-diphenylalanine-lysine, and/or methionine-glycine-lysine, and any derivative thereof.

In some embodiments, the renal enzyme cleavable group comprises methionine-X-lysine, wherein X is a hydrophobic amino acid. In some embodiments, the renal enzyme cleavable group comprises glysine-tyrosine. In some embodiments, the renal enzyme cleavable group comprises glycine-lysine.

In some embodiments, the renal enzyme cleavable group comprises a structure selected from the group consisting of

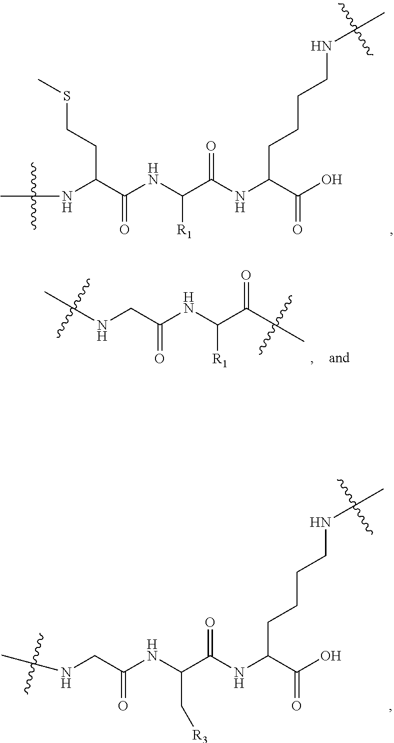

and wherein R₁ is selected from the group consisting of H,

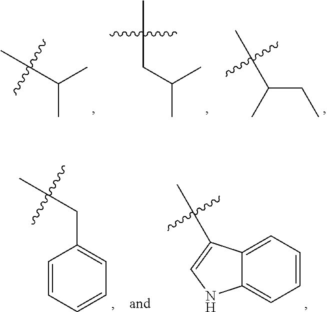

R₂ is selected from the group consisting of H,

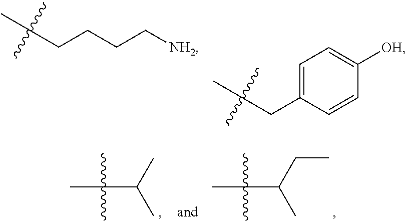

and R₃ is selected from the group consisting of H,

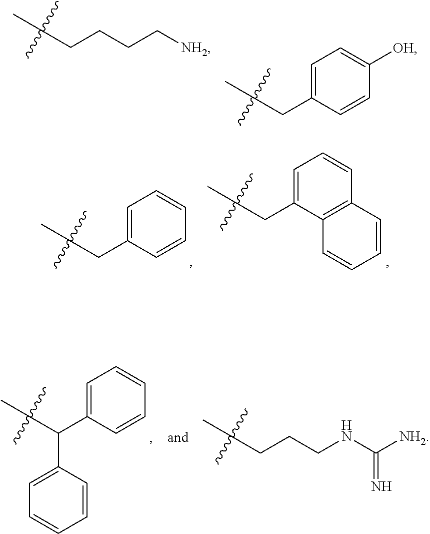

In some embodiments, the functional linker comprises at least one spacer. In some embodiments, the spacer is linear or branched. In some embodiments, the spacer comprises a structure selected from the group consisting of a PEG group as described herein, a C₅₋₂₀ aryl group, a C₁₋₂₀ alkyl group, a C₂₋₂₀ alkenyl group and a C₂₋₂₀ alkynyl group. In some embodiments, the C₅₋₂₀ aryl group, C₁₋₂₀ alkyl group, C₂₋₂₀ alkenyl group or the C₂₋₂₀ alkynyl group is unsubstituted or substituted by one or more substituents selected from the group consisting of N, S, O, Se, P, and halogen atom. In some embodiments, 0, 1 or 2 carbon atoms of the C₅₋₂₀ aryl group, C₁₋₂₀ alkyl group, C₂₋₂₀ alkenyl group or the C₂₋₂₀ alkynyl group are replaced by groups selected from the group consisting of C₆₋₁₀ arylene, 5 to 10 membered heteroarylene group, C₃₋₇ carbocyclylen, 5 to 10 membered heterocyclylene group, and wherein the arylene, heteroarylene, carbocyclylene and heterocyclylene groups are unsubstituted or substituted by one or more substituents selected from the group consisting of N, S, O, Se, P, halogen, C₁₋₆ alkyl, C₁₋₆ alkoxy, C₁₋₆ alkylthiol, —N(C₁₋₆ alkyl) (C₁₋₆ alkyl), nitro and sulfonic acid groups. In some embodiments, 0, 1 or 2-CH— or —CH₂-groups of the C₁₋₂₀ alkyl group, C₂₋₂₀ alkenyl group or the C₂₋₂₀ alkynyl group are replaced by groups selected from the group consisting of —O—, —S—, —S—S—, —C(O)— and —N(C₁₋₆ alkyl)-groups. In some embodiments, the spacer comprises a natural amino acid residue and/or a non-natural amino acid residue. In some embodiments, the natural or non-natural amino acid residue is selected from the group consisting of lysine, aspartate, asparagine, diaminobutyric acid, phenylalanine, tyrosine, threonine, serine, proline, leucine, isoleucine, valine, arginine, histidine, glutamate, glutamine, and alanine.

In some embodiments, the functional linker comprises a structure selected from the group consisting of

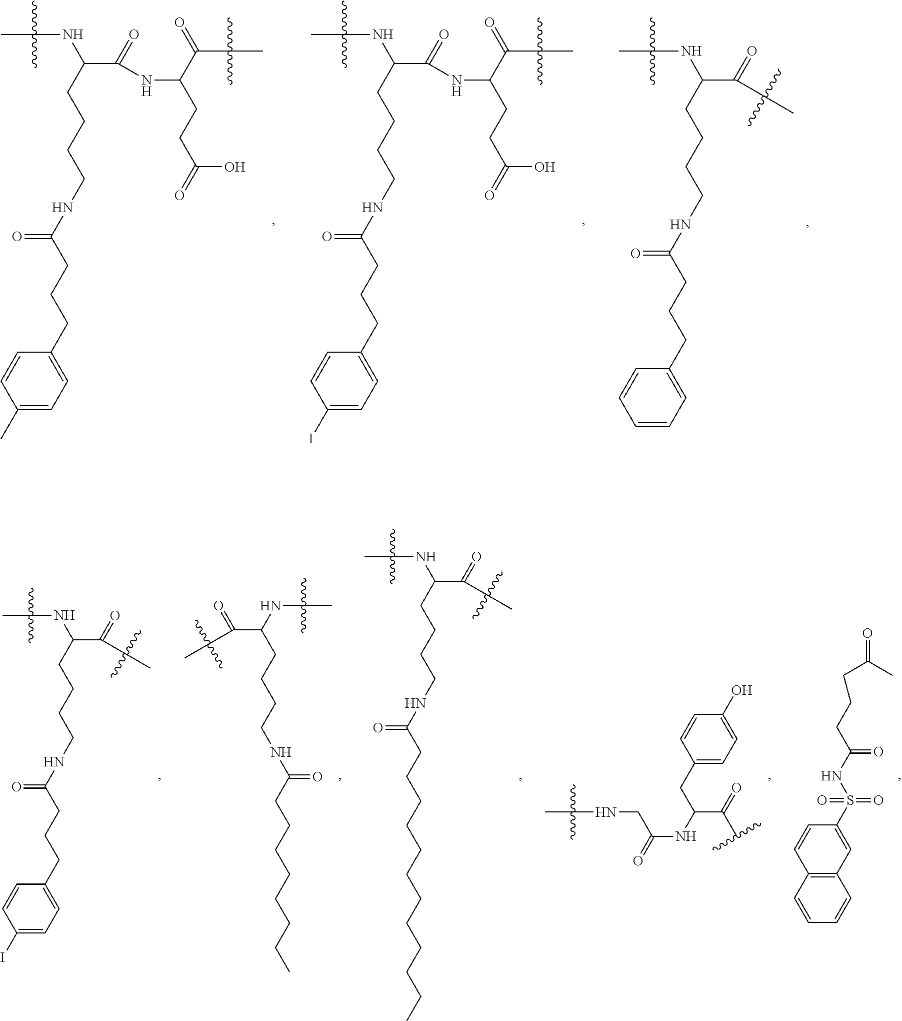

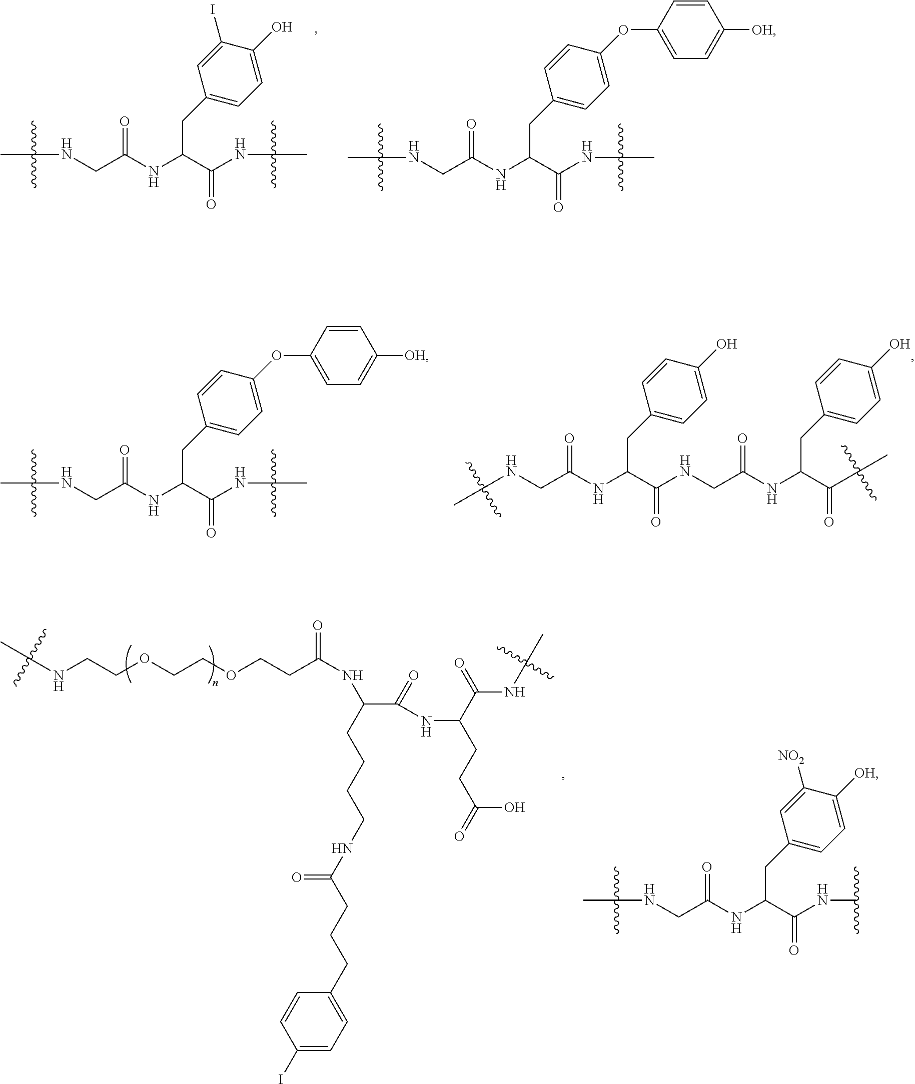

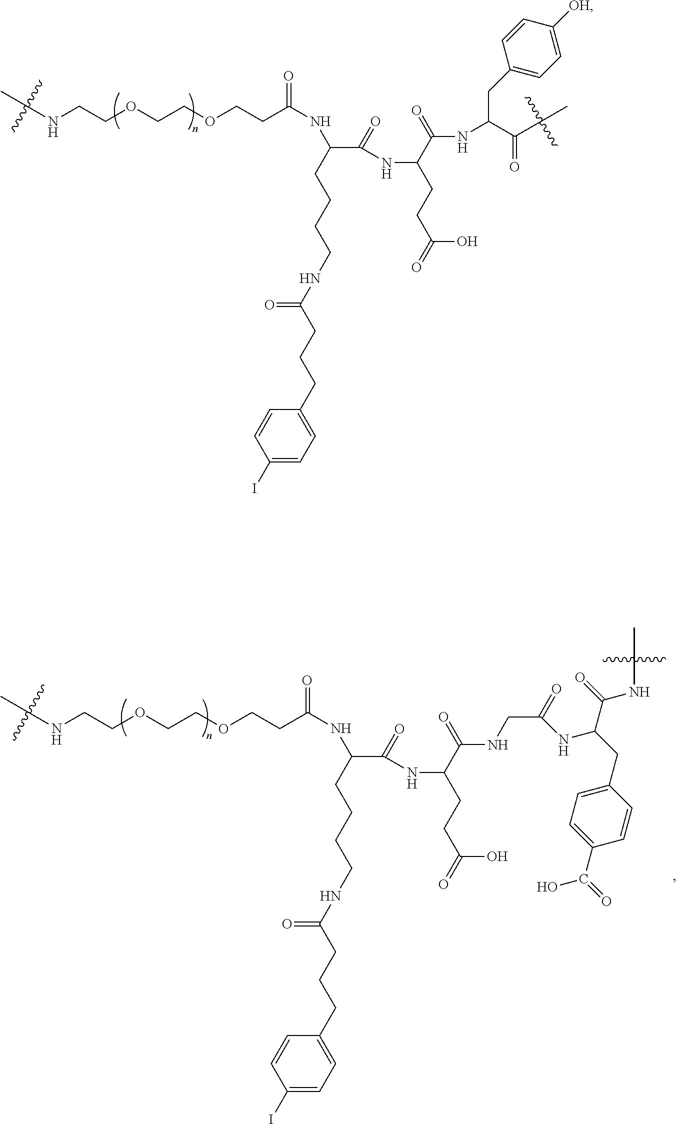

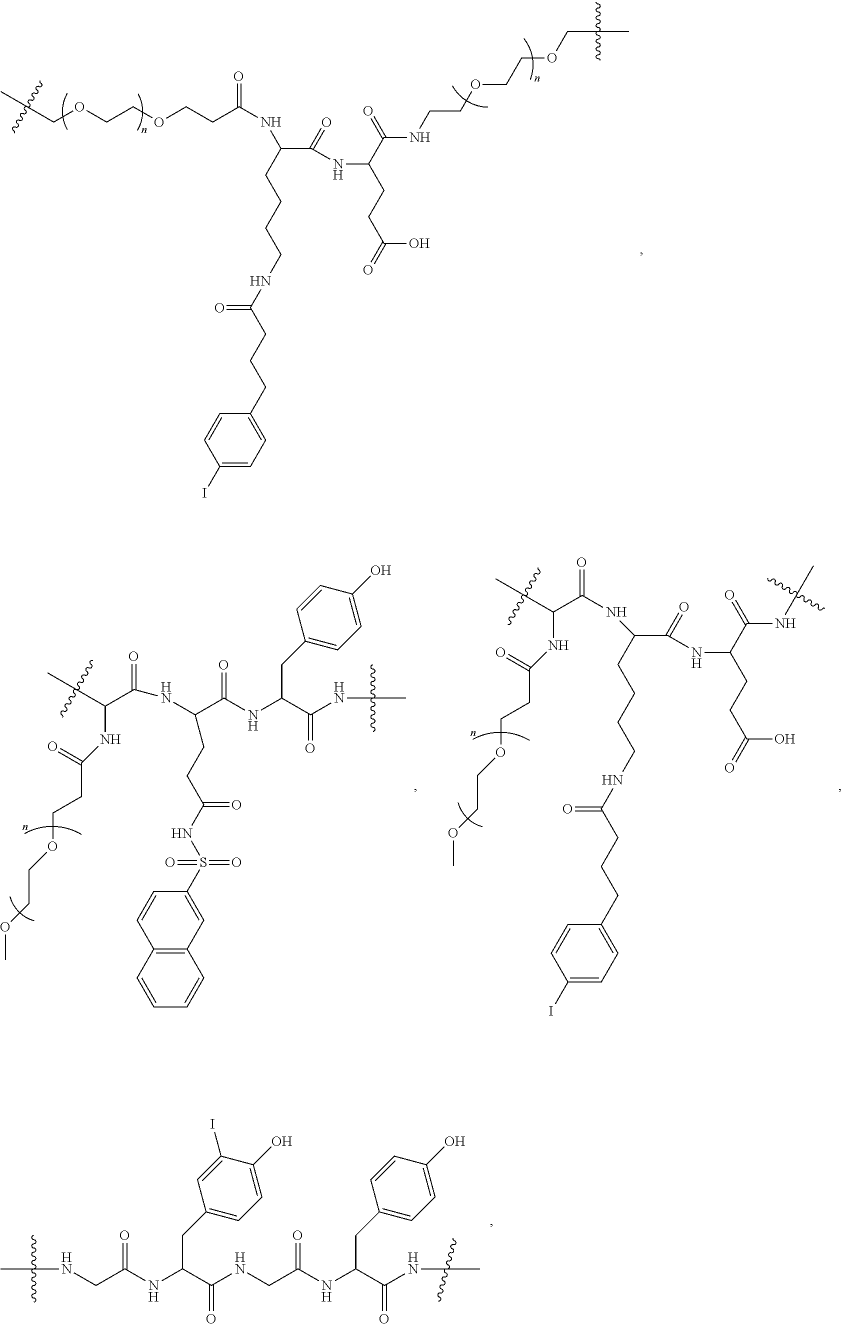

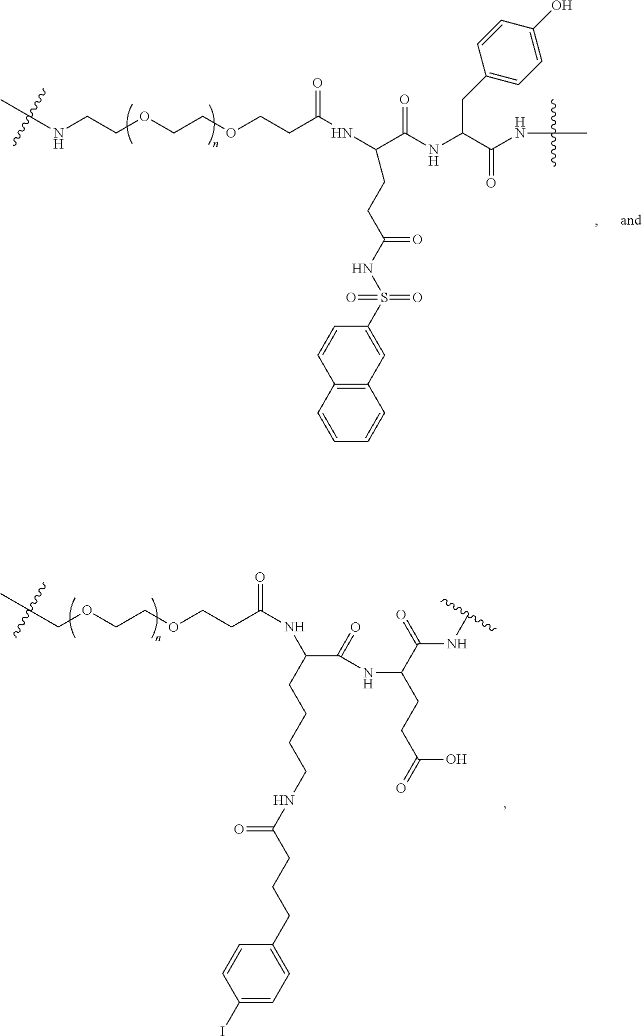

where n is any integer ranging from 2 to 25, including for example any of 2-5, 5-10, 10-15, 15-20, and 20-25.

In some embodiments, the functional linker further comprises a chelator group. Exemplary chelator group can be used in the functional linker of the present application includes but is not limited to NODASA, NODAGA, TETA, TRITA, TRAP, DTPA, CHX-DTPA EDTA, CDTA, CPTA, DOTP, DOTPI, EGTA, HBED, TTHA, DTPA, DOTA, DOTAGA, NOTA, HP-DOA3, CBTE2a, TE2A, TMT, DPDP, HYNIC, DFO, HEDTA, NOPO. MAG3, NCS-MP-NODA, NH2-MPAA-NODA, H2DEDPA, H4octapa, Macropa, Pypa, Py4pa, THP, and SarAr. In some embodiments, the functional linker comprises a DOTA or a DOTAGA group.

In some embodiments, the functional linker comprises a conjugation group capable of connecting the functional linker with the sdAb. In some embodiments, the conjugation group, prior to the conjugation, is selected from the group consisting of an amine, a thiol, an alcohol, a ketone, an aldehyde, a nitrile, a carboxylic acid, an ester, an alkene, an alkyne, an anhydride, a succinimide, a maleimide, a phosphine, a disulfide, an alkoxyamine, an azide, an alkyl halide, an isothiocyanate (NCS), an epoxide, an isocyanate, a hydrazine, and an acyl halide.

In some embodiments, the functional linker comprises a group resulting from the conjugation of any one of the structures provided in Table 2. The functional linker thus may comprise the structure of any of the groups provided below except for the conjugation group.

TABLE 2
Exemplary functional linker structures

| Struc- | |
|---|---|
| ture | |
| Num- | |
| ber | Structure |
| 001 | 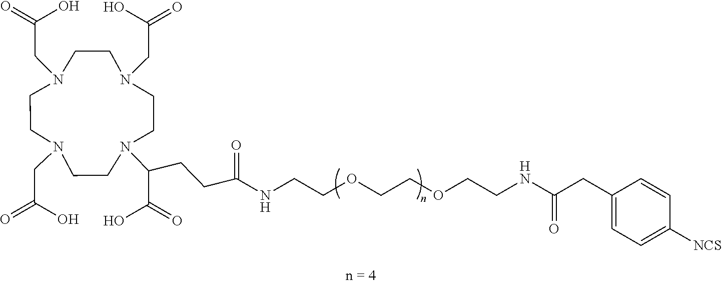 |
| 002 | 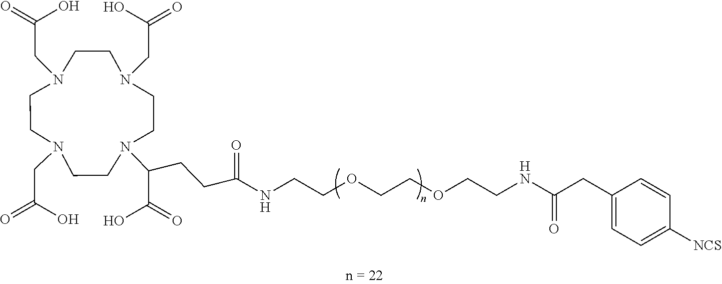 |
| 003 | 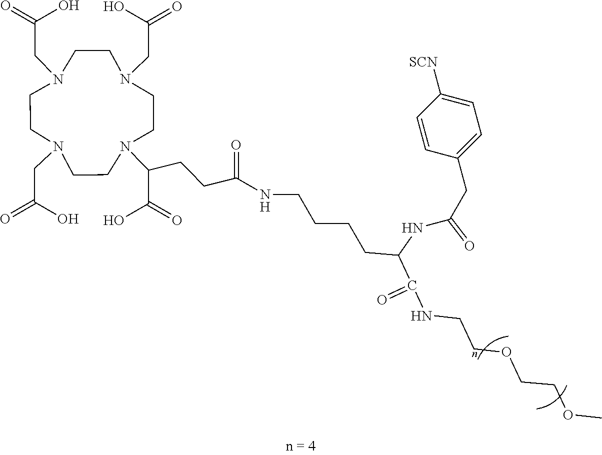 |
| 004 | 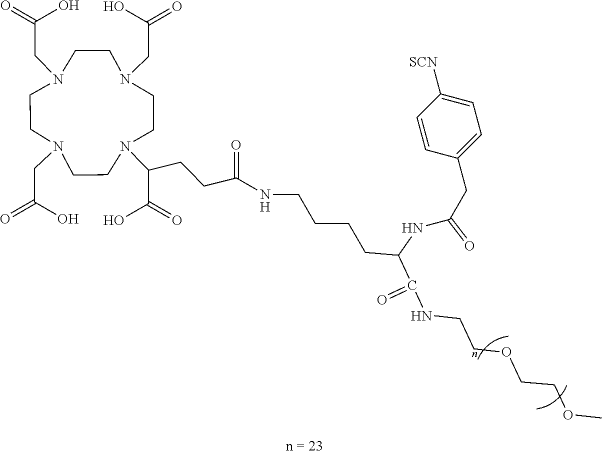 |
| 005 | 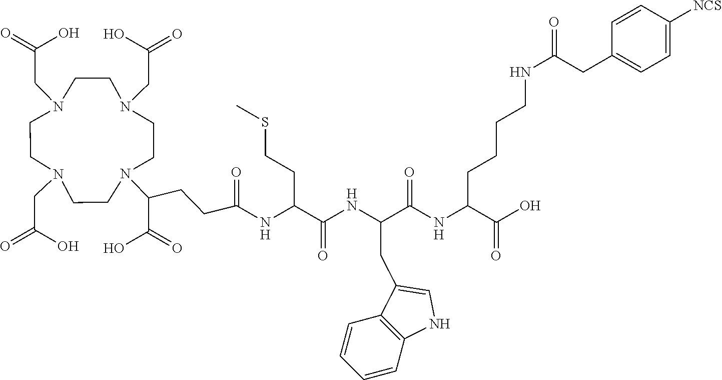 |
| 006 | 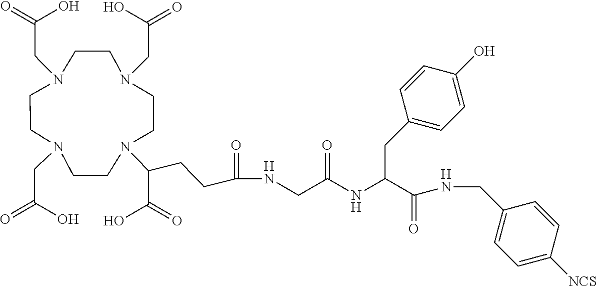 |
| 007 | 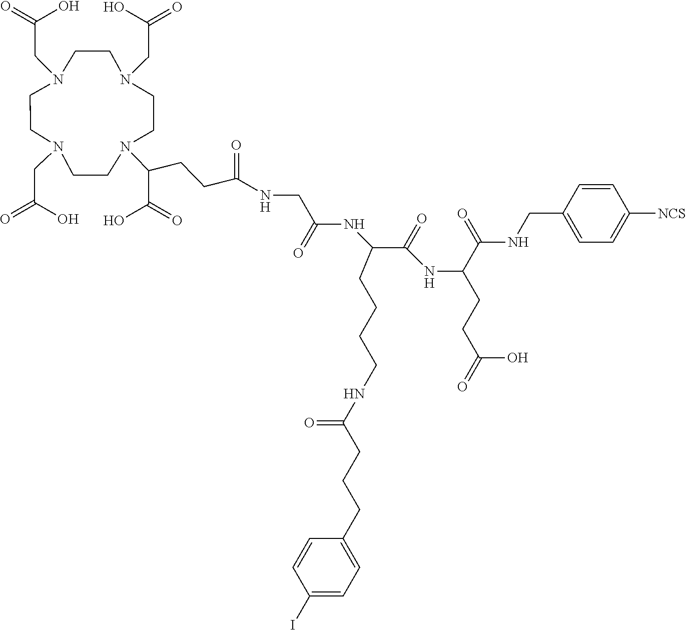 |
| 008 | 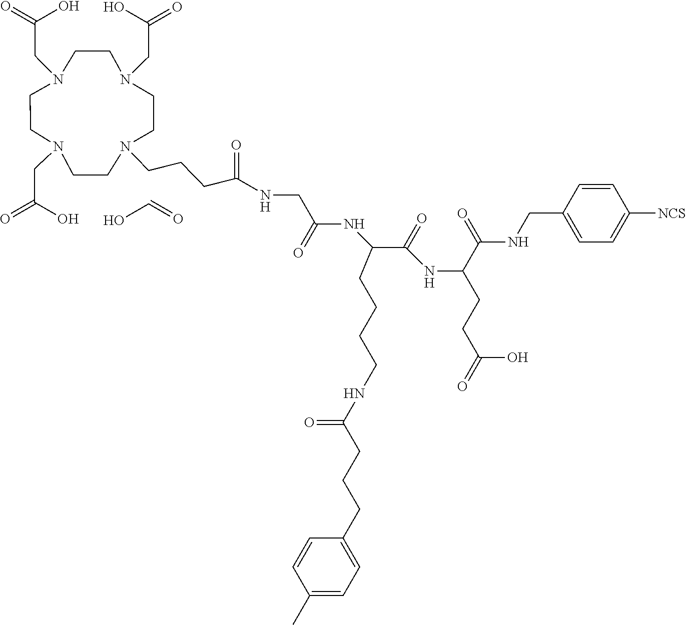 |
| 009 | 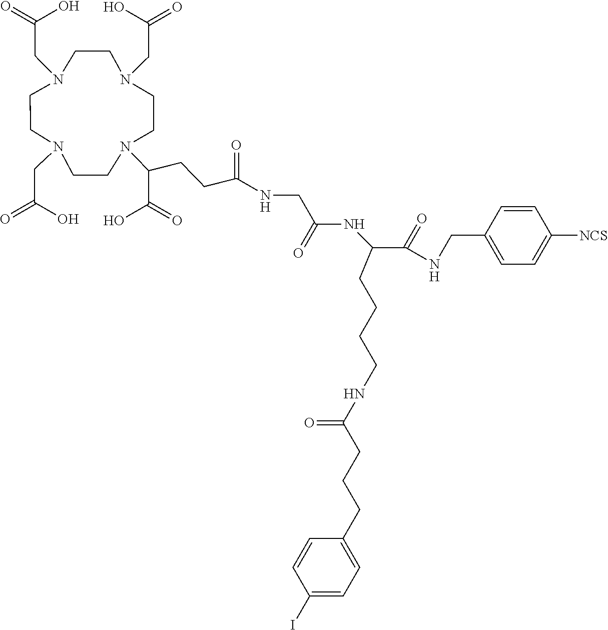 |
| 010 | 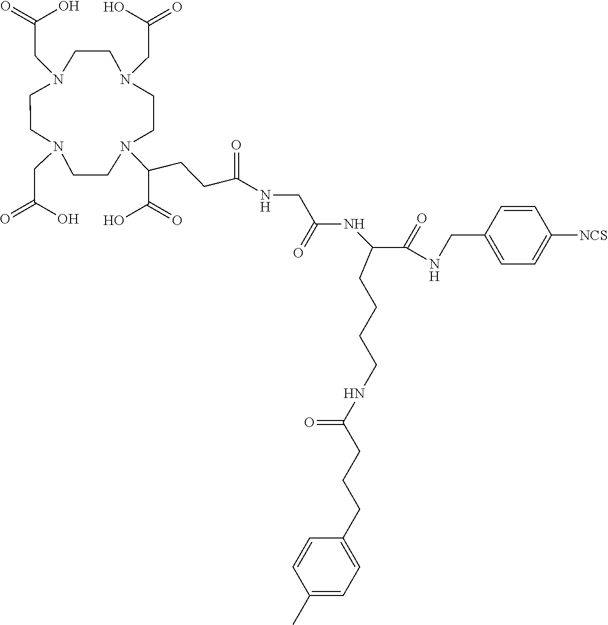 |
| 011 | 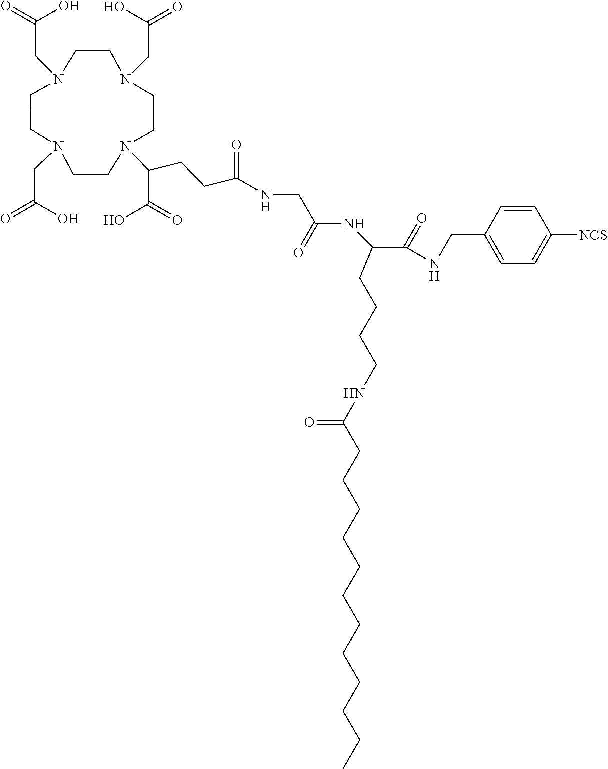 |
| 012 | 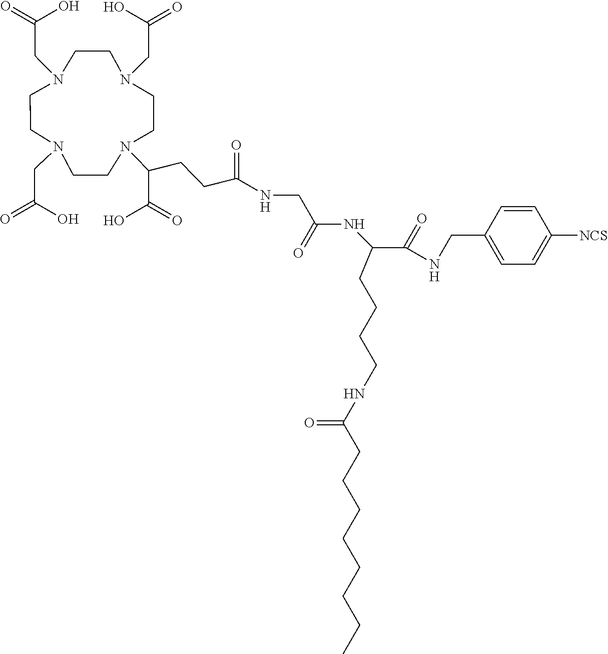 |
| 013 | 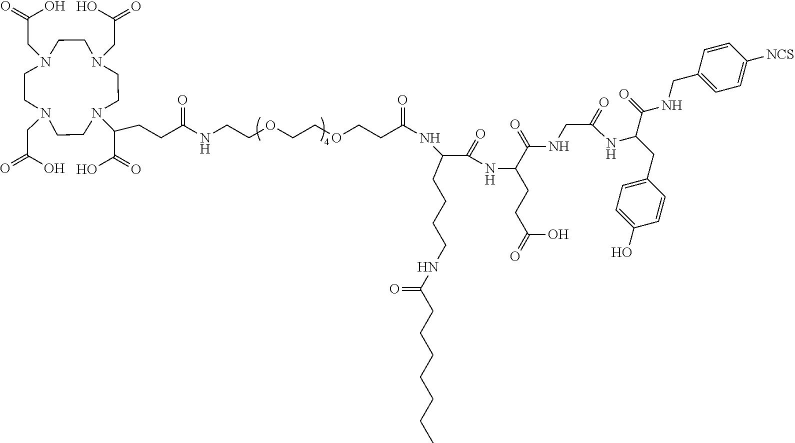 |
| 014 | 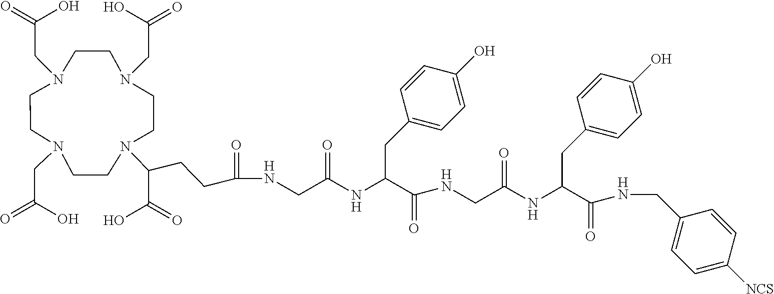 |
| 015 | 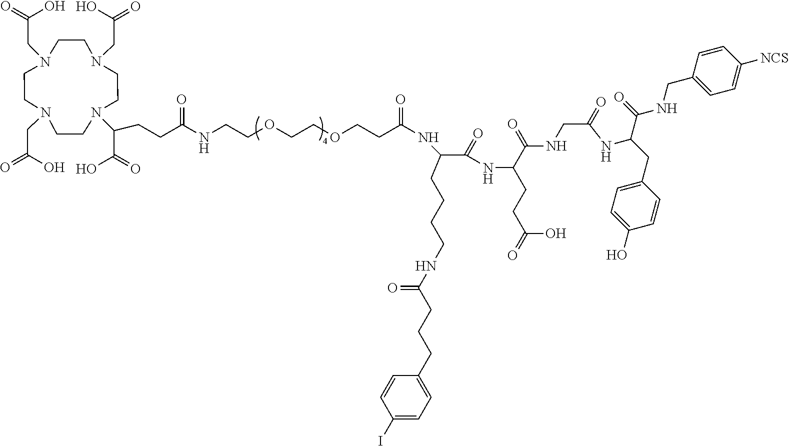 |
| 016 | 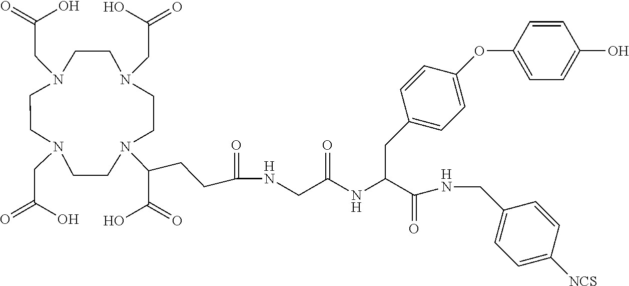 |
| 017 | 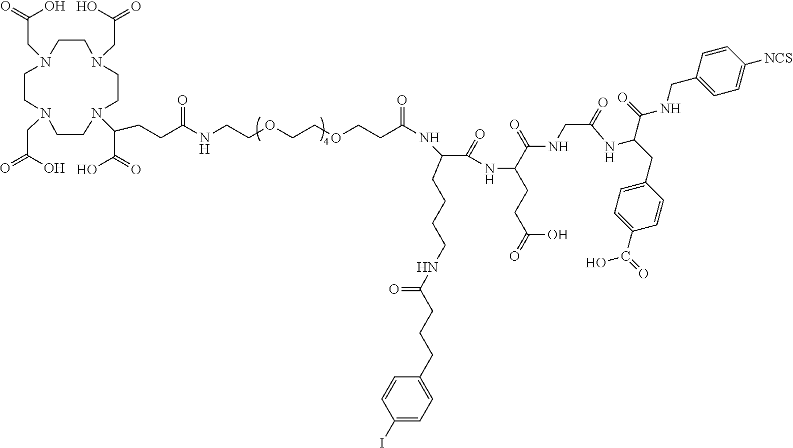 |
| 018 | 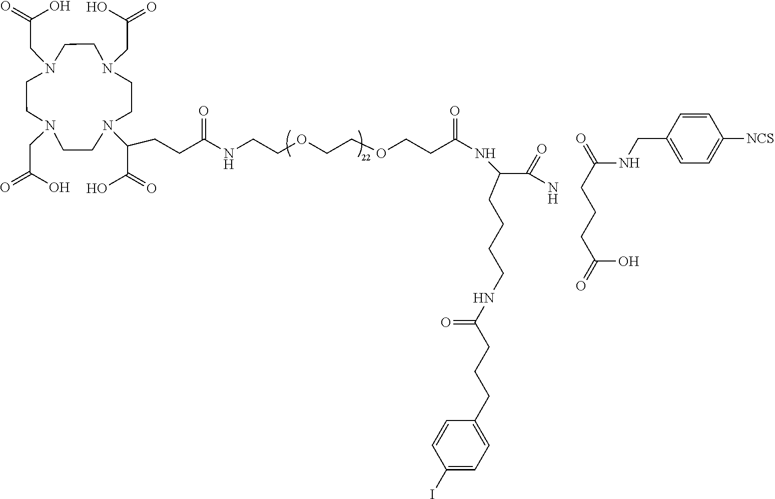 |
| 019 | 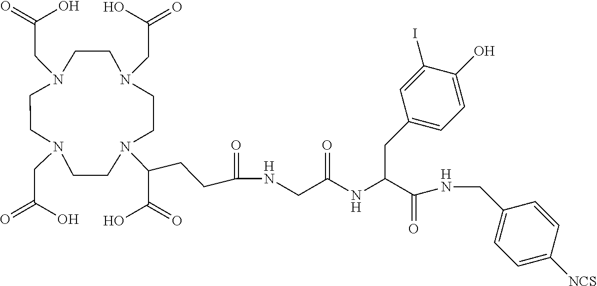 |
| 020 | 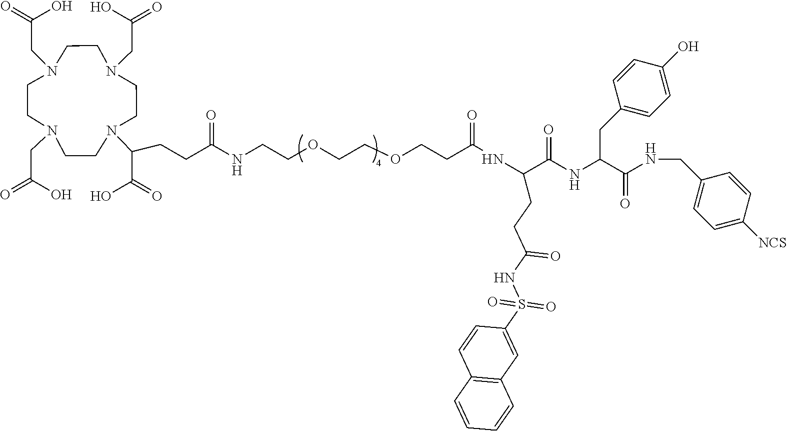 |
| 021 | 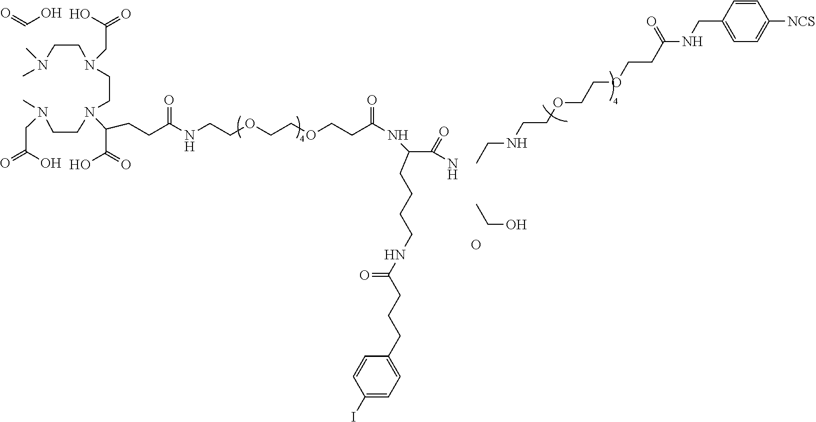 |
| 022 | 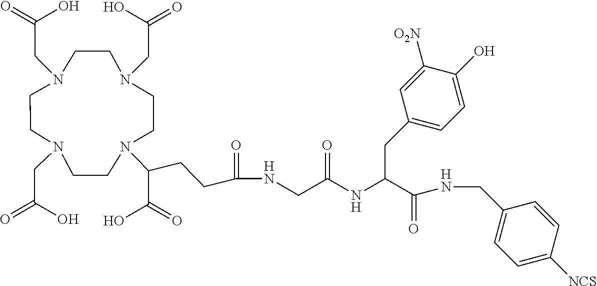 |
| 023 | 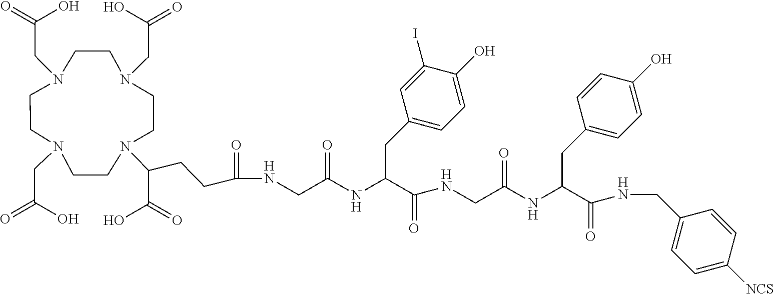 |
| 024 | 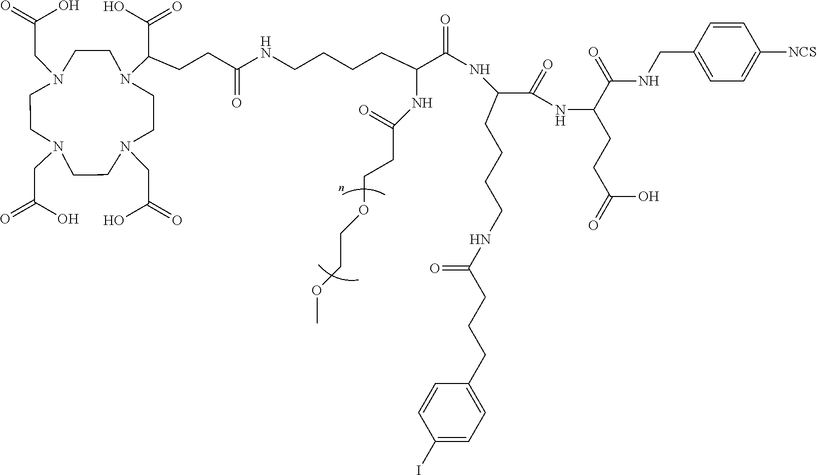 |
| 025 | 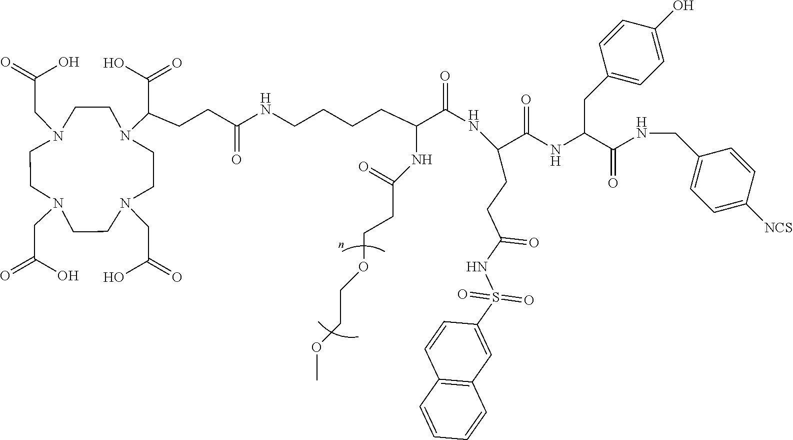 |
| 026 | 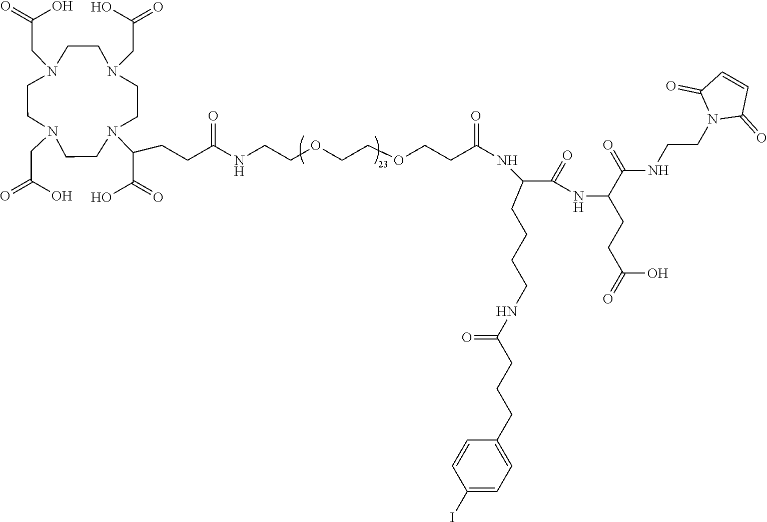 |
| 027 | 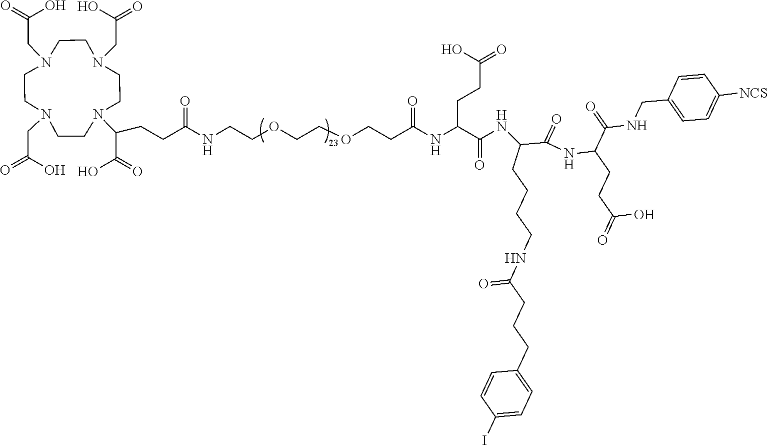 |

Single Domain Antibody (sdAb)

In some embodiments, the conjugate of the present application comprises a single domain antibody (sdAb) that specifically binds to a tumor antigen. In some embodiments, the binding affinity of the sdAb to the tumor antigen is in a range between about 10⁻¹² and about 10⁻⁸ M. In some embodiments, the binding affinity of the sdAb to the tumor antigen is in a range between about 10⁻¹² and about 10⁻¹¹ M. In some embodiments, the binding affinity of the sdAb to the tumor antigen is in a range between about 10⁻¹² and about 10⁻¹⁰ M. In some embodiments, the binding affinity of the sdAb to the tumor antigen is in a range between about 10⁻¹² and about 10⁻⁹ M. In some embodiments, the binding affinity of the sdAb to the tumor antigen is in a range between about 10⁻¹¹ and about 10⁻¹⁰ M. In some embodiments, the binding affinity of the sdAb to the tumor antigen is in a range between about 10⁻¹¹ and about 10⁻⁹ M. In some embodiments, the binding affinity of the sdAb to the tumor antigen is in a range between about 10⁻¹¹ and about 10⁻⁸ M. In some embodiments, the binding affinity of the sdAb to the tumor antigen is in a range between about 10⁻¹⁰ and about 10⁻⁹ M. In some embodiments, the binding affinity of the sdAb to the tumor antigen is in a range between about 10⁻¹⁰ and about 10⁻⁸ M. In some embodiments, the binding affinity of the sdAb to the tumor antigen is in a range between about 10⁻⁹ and about 10⁻⁸ M. In some embodiments, the binding affinity of the sdAb to the tumor antigen is about 10⁻¹² about 10⁻¹¹, about 10⁻¹⁰, about 10⁻⁹ or about 10⁻⁸ M.

In some embodiments, the binding affinity of the conjugate to the tumor antigen is in a range between about 10⁻¹² and about 10⁻⁸ M. In some embodiments, the binding affinity of the conjugate to the tumor antigen is in a range between about 10⁻¹² and about 10⁻¹¹ M. In some embodiments, the binding affinity of the conjugate to the tumor antigen is in a range between about 10⁻¹² and about 10⁻¹⁰ M. In some embodiments, the binding affinity of the conjugate to the tumor antigen is in a range between about 10⁻¹² and about 10⁻⁹ M. In some embodiments, the binding affinity of the conjugate to the tumor antigen is in a range between about 10⁻¹¹ and about 10⁻¹⁰ M. In some embodiments, the binding affinity of the conjugate to the tumor antigen is in a range between about 10⁻¹¹ and about 10⁻⁹ M. In some embodiments, the binding affinity of the conjugate to the tumor antigen is in a range between about 10⁻¹¹ and about 10⁻⁸ M. In some embodiments, the binding affinity of the conjugate to the tumor antigen is in a range between about 10⁻¹⁰ and about 10⁻⁹ M. In some embodiments, the binding affinity of the conjugate to the tumor antigen is in a range between about 10⁻¹⁰ and about 10⁻⁸ M. In some embodiments, the binding affinity of the conjugate to the tumor antigen is in a range between about 10⁻⁹ and about 10⁻⁸ M. In some embodiments, the binding affinity of the conjugate to the tumor antigen is about 10⁻¹², about 10⁻¹¹, about 10⁻¹⁰, about 10⁻⁹ or about 10⁻⁸ M.

In some embodiments, the conjugate of the present application comprises a functional linker and the functional linker comprises an albumin binding group. In some embodiments, the ratio of the binding affinity of the conjugate to the tumor antigen relative to the binding affinity of the conjugate to human serum albumin is between about 10 to about 10⁴. In some embodiments, the ratio of the binding affinity of the conjugate to the tumor antigen relative to the binding affinity of the conjugate to human serum albumin is between about 10 to about 100. In some embodiments, the ratio of the binding affinity of the conjugate to the tumor antigen relative to the binding affinity of the conjugate to human serum albumin is between about 10 to about 1000. In some embodiments, the ratio of the binding affinity of the conjugate to the tumor antigen relative to the binding affinity of the conjugate to human serum albumin is between about 100 to about 1000. In some embodiments, the ratio of the binding affinity of the conjugate to the tumor antigen relative to the binding affinity of the conjugate to human serum albumin is between about 100 to about 10⁴. In some embodiments, the ratio of the binding affinity of the conjugate to the tumor antigen relative to the binding affinity of the conjugate to human serum albumin is between about 1000 to about 10⁴.

In some embodiments, the sdAb of the conjugate specifically binds to a tumor antigen selected from the group consisting of specific membrane antigen (PSMA), fibroblast activation protein alpha (FAP-alpha, FAP), a folate receptor, luteinizing hormone-releasing hormone (LHRH), noradrenaline transporter (NAT), epidermal growth factor receptor (EGFR), Human Epidermal Growth Factor Receptor-2 (HER-2), vascular endothelial growth factor (VGFR), Mucin-1 (MUC-1), Mucin-4 (MUC-4), urokinase-type plasminogen activator receptor (uPAR), tumor-associated glycoprotein 72 (TAG-72), Claudin 18.2 (CLDN18.2), vascular endothelial growth factor receptor (VEGFR), C—X—C chemokine receptor type 4 (CXCR4), Hepsin, Transmembrane protease, serine 2 (TMPRSS2), and tyrosine-protein kinase Met (cMET).

Claudin 18.2 (or CLDN18.2) is a protein that in humans is encoded by the CLDN18 gene. GLDN18.2 is highly expressed in a significant proportion of gastric and pancreatic adenocarcinomas, while normal tissue expression is limited to the epithelium of the stomach. See G. Zhu, et al. (2019) Sci. Rep. 9 (1): 8420.

In some embodiments, the present application provides an anti-CLDN18.2 sdAb comprising the amino acid sequence as set forth in SEQ ID NO: 1, or a variant thereof having at least about 80% (such as at least about any of 80%, 85%, 90%, 91%, 92%, 93%, 94%, 95%, 96%, 97%, 98%, or 99%) sequence identity to SEQ ID NO: 1.

In some embodiments, the sdAb of the conjugate as described herein specifically binds to CLDN18.2 and comprises a CDR1, CDR2 and CDR3 of the sequence as set forth in SEQ ID NO. 1. In some embodiments, the CDR1, CDR2 and CDR3 are according to IMGT numbering. In some embodiments, the CDR1, CDR2 and CDR3 are according to Kabat numbering. In some embodiments, the CDR1, CDR2 and CDR3 are according to Chothia numbering. In some embodiments, the CDR1, CDR2 and CDR3 are according to Abm numbering. In some embodiments, the CDR1, CDR2 and CDR3 are according to Contact numbering.

In some embodiments, the sdAb of the conjugate specifically binds to CLDN18.2 and comprises: (1) a CDR1 comprising an amino acid sequence as set forth in SEQ ID NO. 2 or an amino acid sequence with one or more amino acid alterations as compared to SEQ ID NO. 2, (2) a CDR2 comprising an amino acid sequence as set forth in SEQ ID NO. 3 or an amino acid sequence with one or more amino acid alterations as compared to SEQ ID NO. 3, and (3) a CDR3 comprising an amino acid sequence as set forth in SEQ ID NO. 4 or an amino acid sequence with one or more amino acid alterations as compared to SEQ ID NO. 4. Amino acid alterations include, for example, substitutions, deletions, and insertions. In some embodiments, the sdAb specifically binds to CLDN18.2 and comprises: (1) a CDR1 comprising an amino acid sequence as set forth in SEQ ID NO. 2, (2) a CDR2 comprising an amino acid sequence as set forth in SEQ ID NO. 3, and (3) a CDR3 comprising an amino acid sequence as set forth in SEQ ID NO. 4.

In some embodiments, the sdAb of the conjugate described herein specifically binds to CLDN18.2 and comprises the amino acid sequence as set forth in SEQ ID NO: 1, or a variant thereof having at least about 80% (such as at least about any of 80%, 85%, 90%, 91%, 92%, 93%, 94%, 95%, 96%, 97%, 98%, or 99%) sequence identify to any one of SEQ ID NO:1. In some embodiments, the sdAb specifically binds to CLDN18.2 and comprises the amino acid sequence as set forth in SEQ ID NO: 1, or a variant thereof comprising up to about 10 (such as about any of 1, 2, 3, 4, 5, 6, 7, 8, 9, or 10) amino acid substitutions as compared to SEQ ID NO. 1. In some embodiments, the sdAb specifically binds to CLDN18.2 and comprises the amino acid sequence of SEQ ID NO:1, or a variant thereof comprising up to about 3 (such as about any one of 1, 2, or 3) amino acid substitutions in the CDR1, and/or the CDR2, and/or the CDR3 of SEQ ID NO. 1. In some embodiments, the sdAb specifically binds to CLDN18.2 and comprises the amino acid sequence of SEQ ID NO:1, or a variant thereof comprising up to about 3 (such as about any one of 1, 2, or 3) amino acid substitutions in the FR1, and/or the FR2, and/or the FR3, and/or the FR4 of SEQ ID NO. 1. In some embodiments, the sdAb specifically binds to CLDN18.2 and comprises the amino acid sequence of SEQ ID NO:1, or a variant thereof comprising amino acid substitutions in both CDRs and FRs. In some embodiments, the sdAb specifically binds to CLDN18.2 and comprises the amino acid sequence of SEQ ID NO:1.

Fibroblast activation protein alpha (FAP or FAP-alpha) is a 170 kDa single pass type II transmembrane glycoprotein belonging to the dipeptidyl peptidase 4 family. It is highly expressed in cancer-associated fibroblasts (CAFs) and plays an important role in modulating the tumor microenvironment and supports the tumors cells through the release of enzymes, cytokines and growth factor. FAP can provide target specificity to therapeutic agents because in adult humans its expression is restricted to pathologic sites, including cancer, fibrosis, arthritis, wounding, or inflammation. See T. Kelly, et al. (2012) Int. Rev. Cell. Mol. Biol. 297:83-116. Studies have shown that FAP expression is highly upregulated on reactive CAFs of more than 90% of all primary and metastatic epithelial tumors, while it is generally absent from normal adult tissues. FAP overexpression is associated with poor prognosis and increased risk of metastasis.

In some embodiments, the present application provides an anti-FAP sdAb comprising the amino acid sequence as set forth in SEQ ID NO: 5, or a variant thereof having at least about 80% (such as at least about any of 80%, 85%, 90%, 91%, 92%, 93%, 94%, 95%, 96%, 97%, 98%, or 99%) sequence identity to of SEQ ID NO: 5.

In some embodiments, the sdAb of the conjugate as described herein specifically binds to FAP. In some embodiments, the sdAb of the conjugate specifically binds to FAP and comprises a CDR1, CDR2 and CDR3 of the sequence as set forth in SEQ ID NO. 5. In some embodiments, the CDR1, CDR2 and CDR3 are according to IMGT numbering. In some embodiments, the CDR1, CDR2 and CDR3 are according to Kabat numbering. In some embodiments, the CDR1, CDR2 and CDR3 are according to Chothia numbering. In some embodiments, the CDR1, CDR2 and CDR3 are according to Abm numbering. In some embodiments, the CDR1, CDR2 and CDR3 are according to Contact numbering.

In some embodiments, the sdAb of the conjugate specifically binds to FAP and comprises: (1) a CDR1 comprising an amino acid sequence as set forth in SEQ ID NO. 6 or an amino acid sequence with one or more amino acid alterations as compared to SEQ ID NO. 6, (2) a CDR2 comprising an amino acid sequence as set forth in SEQ ID NO. 7 or an amino acid sequence with one or more amino acid alterations as compared to SEQ ID NO. 7, and (3) a CDR3 comprising an amino acid sequence as set forth in SEQ ID NO. 8 or an amino acid sequence with one or more amino acid alterations as compared to SEQ ID NO. 8. Amino acid alterations include, for example, substitutions, deletions, and insertions. In some embodiments, the sdAb specifically binds to FAP and comprises: (1) a CDR1 comprising an amino acid sequence as set forth in SEQ ID NO. 6, (2) a CDR2 comprising an amino acid sequence as set forth in SEQ ID NO. 7, and (3) a CDR3 comprising an amino acid sequence as set forth in SEQ ID NO. 8.

In some embodiments, the sdAb of the conjugate specifically binds to FAP and comprises: (1) a CDR1 comprising an amino acid sequence as set forth in SEQ ID NO. 9 or an amino acid sequence with one or more amino acid alterations as compared to SEQ ID NO. 9, (2) a CDR2 comprising an amino acid sequence as set forth in SEQ ID NO. 10 or an amino acid sequence with one or more amino acid alterations as compared to SEQ ID NO. 10, and (3) a CDR3 comprising an amino acid sequence as set forth in SEQ ID NO. 11 or an amino acid sequence with one or more amino acid alterations as compared to SEQ ID NO. 11. Amino acid alterations include, for example, substitutions, deletions, and insertions. In some embodiments, the sdAb specifically binds to FAP and comprises: (1) a CDR1 comprising an amino acid sequence as set forth in SEQ ID NO. 9, (2) a CDR2 comprising an amino acid sequence as set forth in SEQ ID NO. 10, and (3) a CDR3 comprising an amino acid sequence as set forth in SEQ ID NO. 11.

In some embodiments, the sdAb of the conjugate described herein specifically binds to FAP and comprises the amino acid sequence as set forth in SEQ ID NO: 5, or a variant thereof having at least about 80% (such as at least about any of 80%, 85%, 90%, 91%, 92%, 93%, 94%, 95%, 96%, 97%, 98%, or 99%) sequence identify to any one of SEQ ID NO:5. In some embodiments, the sdAb specifically binds to FAP and comprises the amino acid sequence as set forth in SEQ ID NO: 5, or a variant thereof comprising up to about 10 (such as about any of 1, 2, 3, 4, 5, 6, 7, 8, 9, or 10) amino acid substitutions as compared to SEQ ID NO. 5. In some embodiments, the sdAb specifically binds to FAP and comprises the amino acid sequence of SEQ ID NO:5, or a variant thereof comprising up to about 3 (such as about any one of 1, 2, or 3) amino acid substitutions in the CDR1, and/or the CDR2, and/or the CDR3 of SEQ ID NO. 5. In some embodiments, the sdAb specifically binds to FAP and comprises the amino acid sequence of SEQ ID NO:5, or a variant thereof comprising up to about 3 (such as about any one of 1, 2, or 3) amino acid substitutions in the FR1, and/or the FR2, and/or the FR3, and/or the FR4 of SEQ ID NO. 5. In some embodiments, the sdAb specifically binds to FAP and comprises the amino acid sequence of SEQ ID NO:5, or a variant thereof comprising amino acid substitutions in both CDRs and FRs. In some embodiments, the sdAb specifically binds to FAP and comprises the amino acid sequence of SEQ ID NO:5.

The various SEQ ID NOs are depicted in Table 3.

TABLE 3
Sequences Listing

| SEQ ID | | |
|---|---|---|
| NO. | Sequence | Description |
| 1 | EVQLVESGGGLVQPGGSLRLSCAASGSFFRIVAMGWYRQAPG | CLDN18.2 sdAb |
| | SQRELVATITRGGSTYYADSVKGRFTISRDNAKNTLYLQMNS | |
| | LRAEDTAVYYCNVRVEVPFMQPNDYWGQGTLVTVSS | |
| 2 | GSFFRIVA | CDR1 |
| 3 | ITRGGST | CDR2 |
| 4 | NVRVEVPFMQPNDY | CDR3 |
| 5 | QVQLLESGGGLVQPGGSLRLSCAASGFGFGNSLMSWVRRAP | FAP sdAb |
| | GKGLEWVSTIYPRGSFTDYADSVKGRFTISRDNSRNTLYLQM | |
| | NSLRAEDTAVYYCATGWGRSSDYDPPGQGTLVTVSS | |
| 6 | GFGFGNSLMS | CDR1 |
| 7 | YPRGSFTDY | CDR2 |
| 8 | TGWGRSSDYDP | CDR3 |
| 9 | GFGFGNSL | CDR1 (IMGT) |
| 10 | IYPRGSFT | CDR2 (IMGT) |
| 11 | ATGWGRSSDYDP | CDR3 (IMGT) |

### Drug

The drug in the conjugate as described herein can be any suitable drug known in the art. In some embodiments, the drug is selected from the group consisting of a chemotherapeutic agent, a toxin, a cytokine, an enzyme, an immunomodulator, a chelating complex, a diagnostic agent, a nanoparticle, and a radioisotope.

In some embodiments, the drug of the conjugate as described herein comprises a chemotherapeutic agent useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, Astrazeneca), Sutent (SUI 1248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, GlaxoSmithKline), Lonafarnib (SCH 66336), Sorafenib (BAY43-9006, Bayer Labs.), and Gefitinib (IRESSA®, Astrazeneca), AG1478, AG1571 (SU 5271; Sugen), alkylating agents such as Thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins; a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N. J.), ABRAXANE™ Cremophor-free, albumin-engineered nanoparticle formulation of paclitaxel (American Pharmaceutical Partners, Schaumberg, Illinois), and TAXOTERER doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluorometlhylornithine (DMFO); retinoids such as retinoic acid; capecitabine; and pharmaceutically acceptable salts, acids or derivatives of any of the above.

In some embodiments, the chemotherapeutic agent also includes but is not limited to (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; (ii) aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in abherant cell proliferation, such as, for example, PKC-alpha, Ralf and H-Ras; (viii) ribozymes such as a VEGF expression inhibitor (e.g., ANGIOZYME® ribozyme) and a HER2 expression inhibitor; (ix) vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; (x) anti-angiogenic agents such as bevacizumab (AVASTIN®, Genentech); and (xi) pharmaceutically acceptable salts, acids or derivatives of any of the above.

In some embodiments, the drug of the conjugate as described herein comprises a cytokine. The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-a and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-a and TGF-β; insulin-like growth factor-I and -II; erythropoietin (EPO); osteoinductive factors; interferons such as interferon-a, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-10, IL-11, IL-12; a tumor necrosis factor such as TNF-a or TNF-β; and other polypeptide factors including LIF and kit ligand (KL).

In some embodiments, the drug of the conjugate as described herein comprises the radioisotope. In some embodiments, the radioisotope can be selected from the group consisting of bismuth-213, caesium-131, caesium-137, c caesium-131, cobalt-60, holmium-166, iodine-125, iodine-131, iridium-192, lead-212, lutetium-177, palladium-103, phosphorus-32, potassium-42, radium-223, rhenium-186, rhenium-188, samarium-153, scandium-47, selenium-75, sodium-24, strontium-89, technetium-99m, thorium-227, xenon-133, ytterbium-169, ytterbium-177, yttrium-90, actinium-225, astatine-211, bismuth-213, carbon-11, nitrogen-13, oxygen-15, fluorine-18, cobalt-57, copper-64, copper-67, gallium-67, gallium-68, indium-111, iodine-123, iodine-124, krypton-81m, rubidium-82, strontium-82, thallium-201, or zirconium-89. In some embodiments, the radioisotope is selected from the group consisting of the group consisting of: actinium-225, fluorine-18, iodine-125, technetium-99m, lutetium-177, indium-111, gallium-68, copper-64, and zirconium-89. In some embodiments, the radioisotope is lutetium-177 or indium-111.

In some embodiments, the drug of the conjugate as described herein comprises a radioisotope bound to a chelator (a chelating complex). In some embodiments, the radioisotope is bound to a chelator selected from the group consisting of NODASA, NODAGA, TETA, TRITA, TRAP, DTPA, CHX-DTPA EDTA, CDTA, CPTA, DOTP, DOTPI, EGTA, HBED, TTHA, DTPA, DOTA, DOTAGA, NOTA, HP-DOA3, CBTE2a, TE2A, TMT, DPDP, HYNIC, DFO, HEDTA, NOPO. MAG3, NCS-MP-NODA, NH2-MPAA-NODA, H2DEDPA, H4octapa, Macropa, Pypa, Py4pa, THP, and SarAr.

### Preparation of the Conjugate

The conjugate of the present application can be prepared by any suitable methods known in the art. For example, the functional linker as described herein can be synthesized by liquid-phase as well as solid-phase synthesis, such as by methods employing 9-fluorenylmethoxycarbonyl (Fmoc) and/or t-butyloxycarbonyl (Boc) chemistries, and/or other synthetic approaches.

Solid-phase synthesis methods and technology are well-established in the art. For example, oligopeptides may be synthesized by sequential incorporation of the amino acid residues of interest one at a time. In such methods, the synthesis is typically initiated by attaching the C-terminal amino acid of the oligopeptides of interest to a suitable resin. Prior to this, reactive side chain and alpha amino groups of the amino acids are protected from reaction by suitable protecting groups, allowing only the alpha carboxyl group to react with a functional group such as an amine group, a hydroxyl group, or an alkyl halide group on the solid support. Following coupling of the C-terminal amino acid to the support, the protecting group on the side chain and/or the alpha amino group of the amino acid is selectively removed, allowing the coupling of the next amino acid of interest. This process is repeated until the desired oligopeptide is fully synthesized, at which point the oligopeptide can be cleaved from the support and purified.

Coupling between each component of the functional linker may require the formation of thioether (—S—) or ether (—O—) linkages and can be achieved either on solid phase or in solution phase. For example, the formation of thioether (—S—) linkage can be achieved by coupling between a thiol-containing compound (such as the thiol group on cysteine side chain) and an alkyl halide (such as 3-(Fmoc-amino) propyl bromide and the like) in an appropriate solvent (such as N,N-dimethylformamide and the like) in the presence of base (such as N,N-diisopropylethylamine and the like). The formation of an ether (—O—) linkage can be achieved via the Mitsunobu reaction between an alcohol (such as the hydroxyl group on the side chain of serine or threonine, for example) and a phenol group (such as the side chain of tyrosine, for example) in the presence of triphenylphosphine and diisopropyl azidicarboxylate (DIAD) in an aprotic solvent (such as 1,4-dioxane and the like). If the reactions are carried out in solution phase, the reactants used are preferably in equivalent molar ratio (1 to 1), and the desired products can be purified by column chromatography or high performance liquid chromatography (HPLC). If the reactions are carried out on solid phase, meaning one reactant has been attached to a solid phase, then the other reactant is normally used in excess amount (³ 3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

In some embodiments, each component of the functional linker of the present application further comprises a reactive linking group capable of joining one component of the functional linker to another via, for example, the preparation method as described herein. In some embodiments, the components include but are not limited to PEG group, a renal enzyme cleavable group, a blood protein binding group, a spacer, and chelator group. In some embodiments, the reactive linking group includes but is not limited to amine, imine, carboxylate, alcohol, thiol, selenol, phenol, ester, acetone, aldehyde, carbene, sulfonyl halide, imidate, anhydride, disulfide, maleimide, phosphine, disulfide, alkoxyamine, azide, alkyne, strained alkyne, strained alkene, halogen, sulfonate, haloacetyl, hydrazide, diazirine, phosphine, tetrazine, isothiocyanate, and oxaziridine.

In some embodiments, the PEG group used in the functional linker comprises a reactive linking group capable of joining the PEG group to the other components of the functional linker. In some embodiments, the PEG group comprises two reactive linking groups capable of joining the PEG group to two other components of the functional linker. Exemplary reactive linking group of the PEG group includes but is not limited to amine, imine, carboxylate, alcohol, thiol, selenol, phenol, ester, acetone, aldehyde, carbene, sulfonyl halide, imidate, anhydride, disulfide, maleimide, phosphine, disulfide, alkoxyamine, azide, alkyne, strained alkyne, strained alkene, halogen, sulfonate, haloacetyl, hydrazide, diazirine, phosphine, tetrazine, isothiocyanate, and oxaziridine.

In some embodiments, the renal enzyme cleavable group used in the functional linker comprises a reactive linking group capable of joining the renal enzyme cleavable group to the other components of the functional linker. In some embodiments, the renal enzyme cleavable group comprises two reactive linking groups capable of joining the renal enzyme cleavable group to two other components of the functional linker. Exemplary reactive linking group of the renal enzyme cleavable group includes but is not limited to amine, imine, carboxylate, alcohol, thiol, selenol, phenol, ester, acetone, aldehyde, carbene, sulfonyl halide, imidate, anhydride, disulfide, maleimide, phosphine, disulfide, alkoxyamine, azide, alkyne, strained alkyne, strained alkene, halogen, sulfonate, haloacetyl, hydrazide, diazirine, phosphine, tetrazine, isothiocyanate, and oxaziridine.

In some embodiments, the blood protein binding group such as the albumin binding group used in the functional linker comprises a reactive linking group capable of joining the blood protein binding group such as the albumin binding group to the other components of the functional linker. In some embodiments, the blood protein binding group such as the albumin binding group comprises two reactive linking groups capable of joining the blood protein binding group such as the albumin binding group to two other components of the functional linker. Exemplary reactive linking group of the blood protein binding group such as the albumin binding group includes but is not limited to amine, imine, carboxylate, alcohol, thiol, selenol, phenol, ester, acetone, aldehyde, carbene, sulfonyl halide, imidate, anhydride, disulfide, maleimide, phosphine, disulfide, alkoxyamine, azide, alkyne, strained alkyne, strained alkene, halogen, sulfonate, haloacetyl, hydrazide, diazirine, phosphine, tetrazine, isothiocyanate, and oxaziridine.

In some embodiments, the chelator group used in the functional linker comprises a reactive linking group capable of joining the chelator group to the other components of the functional linker. In some embodiments, the chelator group comprises two reactive linking groups capable of joining the chelator group to two other components of the functional linker. Exemplary reactive linking group of the chelator group includes but is not limited to amine, imine, carboxylate, alcohol, thiol, selenol, phenol, ester, acetone, aldehyde, carbene, sulfonyl halide, imidate, anhydride, disulfide, maleimide, phosphine, disulfide, alkoxyamine, azide, alkyne, strained alkyne, strained alkene, halogen, sulfonate, haloacetyl, hydrazide, diazirine, phosphine, tetrazine, isothiocyanate, and oxaziridine.

In some embodiments, the spacer used in the functional linker comprises a reactive linking group capable of joining the spacer to the other components of the functional linker. In some embodiments, the spacer comprises two reactive linking groups capable of joining the spacer to two other components of the functional linker. Exemplary reactive linking group of the spacer includes but is not limited to amine, imine, carboxylate, alcohol, thiol, selenol, phenol, ester, acetone, aldehyde, carbene, sulfonyl halide, imidate, anhydride, disulfide, maleimide, phosphine, disulfide, alkoxyamine, azide, alkyne, strained alkyne, strained alkene, halogen, sulfonate, haloacetyl, hydrazide, diazirine, phosphine, tetrazine, isothiocyanate, and oxaziridine.

In some embodiments, the functional linker comprises a conjugation group capable of connecting the functional linker with the sdAb. In some embodiments, the conjugation group is selected from an amine, a thiol, an alcohol, a ketone, an aldehyde, a nitrile, a carboxylic acid, an ester, an alkene, an alkyne, an anhydride, a succinimide, a maleimide, a phosphine, a disulfide, an alkoxyamine, an azide, an alkyl halide, an isothiocyanate (NCS), an epoxide, an isocyanate, a hydrazine, and an acyl halide.

In some embodiments, the functional linker can comprise, starting from the site of attachment to the sdAb: optional space—the PEG group—optional spacer—the renal enzyme cleavable group. In some embodiments, the functional linker can comprise, starting from the site of attachment to the sdAb: optional space—the renal enzyme cleavable group—optional spacer—the PEG group. In some embodiments, the functional linker can comprise, starting from the site of attachment to the sdAb: optional space—the PEG group—optional spacer—the blood protein binding group. In some embodiments, the functional linker can comprise, starting from the site of attachment to the sdAb: optional space—the renal enzyme cleavable group—optional spacer—the blood protein binding group. In some embodiments, the functional linker can comprise, starting from the site of attachment to the sdAb: optional space—the PEG group—optional spacer—the renal enzyme cleavable group—optional spacer—the blood protein binding group. In some embodiments, the functional linker can comprise, starting from the site of attachment to the sdAb: optional space—the renal enzyme cleavable group—optional spacer—the PEG group—optional spacer—the blood protein binding group.

The functional linker can be conjugated to the sdAb via any suitable method known in the art. In some embodiments, the functional linker is conjugated to the sdAb via an amino acid of the sdAb. In some embodiments, the amino acid may be cysteine, lysine, histidine, aspartate, glutamate, arginine, tyrosine, or serine. In other embodiments, the functional linker is conjugated to the sdAb through a chemical group. The chemical group may be an amine, a thiol, an alcohol, a ketone, an aldehyde, a nitrile, a carboxylic acid, an ester, an alkene, an alkyne, an anhydride, a succinimide, a maleimide, a phosphine, a disulfide, an alkoxyamine, an azide, an alkyl halide, an isothiocyanate, an epoxide, an isocyanate, a hydrazine, or an acyl halide.

### Method of Treatment and Uses

In some embodiments, a method is provided for treating a disorder in the subject in need thereof. The method includes administrating an effective amount of conjugate of the present application to the subject. In some embodiments, the disorder is cancer.

Exemplary cancer that can be treated by the conjugate of the present application includes but is not limited to prostate cancer, gastric cancer, colorectal cancer, pancreatic cancer, ovarian cancer, endometrial cancer, non-small cell lung cancer, breast cancer, thyroid cancer, neuroendocrine cancer, endocrine cancer, esophageal cancer, or leukemia. In some embodiments, the cancer is selected from the group consisting of colorectal cancer, esophageal cancer, gastric cancer, pancreatic cancer, breast cancer, and lung cancer.

In some embodiments, the disease is a disease associated with the tumor antigen expression. For example, in some embodiments, the disease (such as cancer) is associated with CLDN18.2. In some embodiments, the disease (such as cancer) is associated with FAP.

In some embodiment, there is provided use of any of the conjugates described herein for the manufacture of medicament of treating a disease (such as cancer, for example any of the cancer described herein).

In some embodiments, there is provided a method of imaging a tumor in a subject, comprising administering to the subject (such as human subject) an effective amount of any one of the conjugates described herein. In some embodiments, there is provided a conjugated described herein for use in imaging a tumor in a subject. In some embodiments, there is provided use of any of the conjugate described herein for the manufacture of a medicament for imaging a tumor in a subject.

### EXAMPLES

In order that this disclosure may be more fully understood, the following Examples are set forth. It should be understood that these examples are for illustrative purposes only and are not to be construed as limiting this disclosure in any manner.

### Example 1: Synthesis of Conjugates of the Present Application

### 1.1 Synthesis of the Functional Linkers

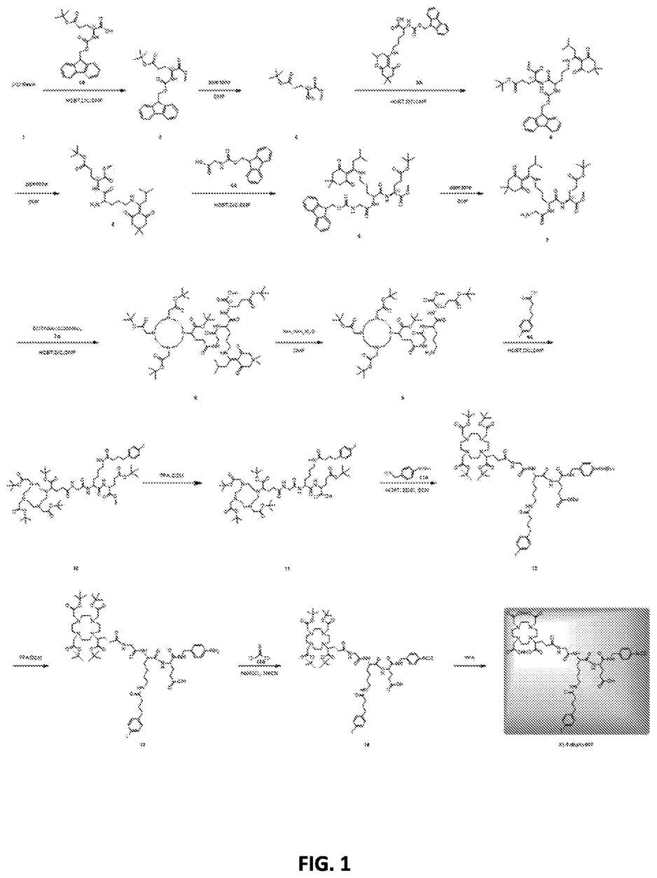

The linkers of the present application were synthesized via solution phase or solid phase methods and further characterized by analytical HPLC and LCMS, respectively. Taking L007 as an example, the linker was synthesized via the scheme as illustrated in FIG. 1. Additional linkers were synthesized based on similar technique. The analytical data is summarized in Table 4 below.

TABLE 4
Characterization of the exemplary synthesized functional linkers

| Linker No. | MW | LCMS | HPLC Purity |
|---|---|---|---|
| L001 | 913.41 | [M + 2H]2+: 457.9 | 98.52% |
| L002 | 1705.88 | [M + 2H]2+: 853.94 | 98.95% |
| L003 | 1012.48 | [M + H]+: 1013.9. | 97.49% |
| L004 | 1848.98 | [M + 3H]3+: 617.9. | 95.81% |
| L005 | 1096.44 | [M + H]+: 1097.9. | 100.00% |
| L006 | 842.33 | [M + H]+: 843.7. | 99.694% |
| L007 | 1208.37 | [M + 2H]2+: 605.7. | 98.377% |
| L008 | 1096.49 | [M + H]+: 1097.9. | 99.681% |
| L009 | 1079.33 | [M + H]+: 1080.7. | 96.615% |
| L010 | 967.45 | [M + H]+: 968.8. | 99.198% |
| L011 | 1003.54 | [M + H]+: 1004.9. | 100.000% |
| L012 | 947.48 | [M + H]+: 948.8. | 99.843% |

### 1.2 Preparation of the SdAb-Linker Conjugates

Claudin 18.2 sdAb (SEQ ID NO. 1, CLDN18.2) and FAP sdAb (SEQ ID NO. 5, FAP) were used as exemplary sdAb to prepare the conjugates. Briefly, CLDN18.2 sdAb and FAP sdAb were treated with 1 mM EDTA in 1×PBS for 30 min at room temperature followed by desalting using PD-10 (Cytiva, #17085101) column to remove trace metal ions, and then buffer exchanging to 50 mM carbonate-bicarbonate (pH 9.5).

Predetermined amount (3× e.q. of sdAb samples) of DOTAGA (used as control for chelating imaging radioisotope) and linkers were diluted in the carbonate-bicarbonate buffer (pH 9.5) and then respectively mixed with the sdAb sample solution with an antibody concentration of 0.5-2 mg/mL. The mixture was incubated at 25° C. for 16 hrs. Then the conjugate was purified using the PD-10 column to remove un-conjugated linkers, and followed by Amicon ultrafiltration unit (Merck, #UFC901024) to further remove the un-conjugated linkers and exchange buffer into 1×PBS. The purified conjugate was then analyzed through SEC-HPLC, and 100 mM PB buffer (5 g NaH₂PO₄ and 25 g Na₂HPO₄ in 1 L of Milli-Q water) was used as the mobile phase. The samples were further subject to LC-MS analysis. The DOL (Degree of Ligation) were calculated based on the LC-MS results, as shown in Table 5 below.

TABLE 5
DOL of the sdAb-Linker conjugates

| SdAb-Linker conjugate | DOL |
|---|---|
| CLDN18.2-DOTAGA | 1.23 |
| CLDN18.2-L001 | 1.13 |
| CLDN18.2-L002 | 1.18 |
| CLDN18.2-L004 | 1.37 |
| CLDN18.2-L007 | 1.69 |
| CLDN18.2-L009 | 1.77 |
| CLDN18.2-L010 | 1.54 |
| CLDN18.2-L011 | 1.71 |
| CLDN18.2-L012 | 1.65 |
| FAP-DOTAGA | 0.42 |
| FAP-L007 | 0.68 |

### Example 2: Binding Affinities of the Conjugates

Binding affinities of the prepared conjugates with tumor antigen was evaluated by ELISA. Briefly, 60 ng/100 μL human Claudin 18.2-VLP or 50 ng/100 μL human FAP-hFc in DPBS buffer was coated onto an ELISA plate and incubated at 4° C. overnight. On the next day, the plate was washed by 0.05% PBST and blocked by 10% StartingBlock buffer (Thermo, #37538) at room temperature for 1 hr. Then, 100 μL of serial diluted conjugate samples in 1% StartingBlock buffer was added to the plate. The plate was further incubated at room temperature for 2 hrs under shaking at 100 rpm and washed by 0.05% PBST. Then 100 μL 0.4 μg/mL HRP-conjugated anti-sdAb secondary antibody (Jackson, #128-035-232) was added and subject to 1 hr incubation at room temperature under shaking at 100 rpm. The plate was then washed by 0.05% PBST and developed using TMB solution (KPL, #5120-00820). The absorbance was read at 450 nm and EC₅₀ of each conjugate was analyzed, as shown in Table 6. From Table 6, the conjugates displayed comparable binding affinity with the CLDN18.2 sdAb or FAP sdAb conjugated with DOTAGA.

TABLE 6
Binding affinities of the conjugates

| SdAb-Linker conjugate | EC50 (nM) |
|---|---|
| CLDN18.2-DOTAGA | 0.84 |
| CLDN18.2-L001 | 1.75 |
| CLDN18.2-L002 | 2.18 |
| CLDN18.2-L004 | 3.47 |
| CLDN18.2-L007 | 7.41 |
| CLDN18.2-L009 | 3.11 |
| CLDN18.2-L010 | 2.31 |
| CLDN18.2-L012 | 1.93 |
| FAP-DOTAGA | 0.05527 |
| FAP-L007 | 0.06029 |

### Example 3: Pharmacokinetic Study of the CLDN18.2-Linker Conjugates

The in vivo pharmacokinetic profile of the CLDN18.2-linker conjugates was analyzed. Briefly, BALB/c mice were administered with the conjugates by intravenous injection at a dose of about 1 mg/kg. Then, whole blood of the animal was collected at different time points over 48 hours.

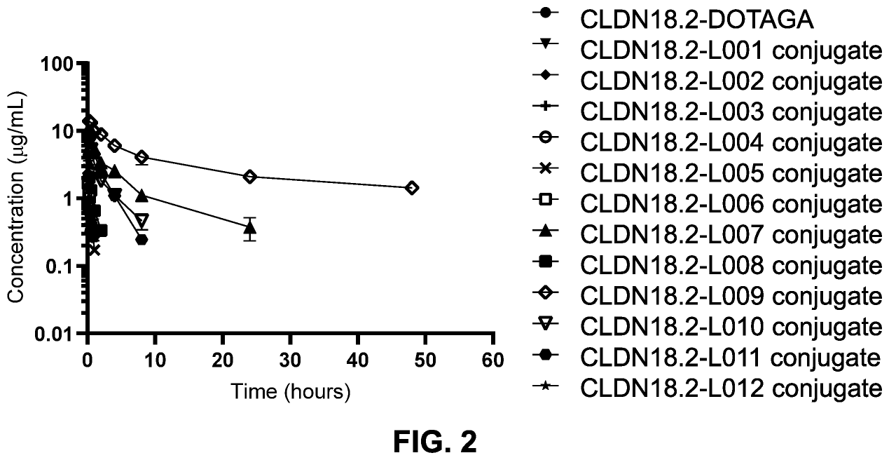

The whole blood was collected into a tube without anticoagulation and maintained at room temperature for more than 30 minutes, followed by centrifuge at approximately 5000 rpm at 4° C. for 5 minutes to obtain serum. Concentration of the conjugates in serum was then measured by ELISA using HRP-labeled goat anti-alpaca IgG VHH domain secondary antibody. The data was further analyzed using PK Solver software, and the non-atrioventricular model (NCA) was used to evaluate the pharmacokinetic parameters, as shown in Table 7 and FIG. 2. FIG. 2 shows that conjugation with the linker 007, linker 009, linker 010 and linker 011 significantly prolonged blood circulation of the CLDN18.2 conjugates as compared to the control group.

TABLE 7
Pharmacokinetic profile of CLDN18.2 conjugate

| | | | AUCₐₗₗ | |
|---|---|---|---|---|
| SdAb-Linker conjugate | T_{1/2} (hr) | V_{d} (L/kg) | (μg/mL hr) | Cl (L/hr/kg) |
| CLDN18.2-DOTAGA | 0.549 ± 0.042 | 0.245 ± 0.032 | 1.45 ± 0.149 | 0.610 ± 0.072 |
| CLDN18.2-L003 | 0.186 ± 0.026 | 0.431 ± 0.006 | 0.460 ± 0.077 | 1.81 ± 0.246 |
| CLDN18.2-L005 | 0.223 ± 0.026 | 0.284 ± 0.017 | 0.881 ± 0.118 | 1.07 ± 0.120 |
| CLDN18.2-L006 | 0.225 ± 0.006 | 0.408 ± 0.026 | 0.650 ± 0.069 | 1.36 ± 0.132 |
| CLDN18.2-L007 | 6.01 ± 1.23 | 0.183 ± 0.014 | 33.1 ± 4.92 | 0.027 ± 0.003 |
| CLDN18.2-L008 | 0.637 ± 0.067 | 0.375 ± 0.043 | 1.88 ± 0.177 | 0.439 ± 0.022 |
| CLDN18.2-L009 | 24.9 ± 5.38 | 0.166 ± 0.023 | 148 ± 5.14 | 0.005 ± 0.000 |
| CLDN18.2-L010 | 2.92 ± 0.117 | 0.199 ± 0.012 | 14.5 ± 0.149 | 0.061 ± 0.002 |
| CLDN18.2-L011 | 1.50 ± 0.168 | 0.106 ± 0.008 | 15.7 ± 0.816 | 0.057 ± 0.001 |
| CLDN18.2-L012 | 0.289 ± 0.019 | 0.151 ± 0.025 | 2.22 ± 0.223 | 0.421 ± 0.042 |

### Example 4: Pharmacokinetic Study of FAP-Linker Conjugates

he in vivo pharmacokinetic profile of the FAP-linker conjugates was analyzed. Briefly, BALB/c mice were administered with the conjugates by intravenous injection at a dose of about 1 mg/kg. Then, whole blood of the animal was collected at different time periods over 48 hours.

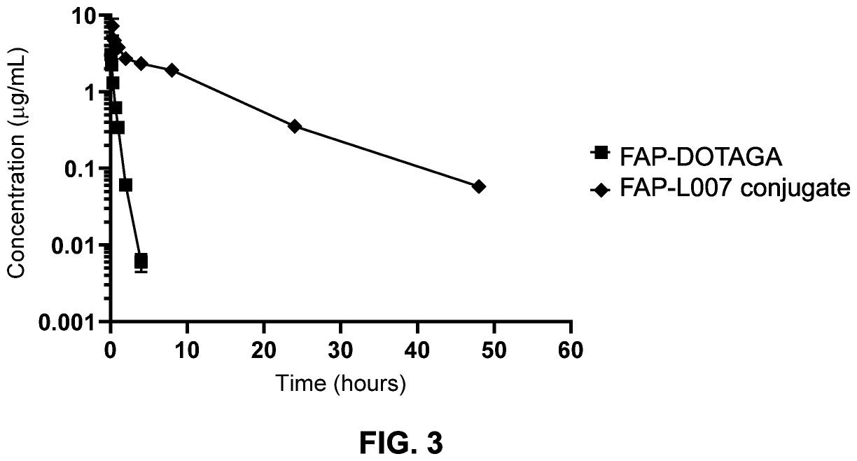

The whole blood was collected into a tube without anticoagulation and maintained at room temperature for more than 30 minutes, followed by centrifugated at approximately 5000 rpm at 4° C. for 5 minutes to obtain serum. Concentration of the conjugates in serum was then measured by ELISA using HRP-labeled goat anti-alpaca IgG VHH domain secondary antibody. The data was further analyzed using PK Solver software, and the non-atrioventricular model (NCA) was used to evaluate the pharmacokinetic parameters, as shown in Table 8 and FIG. 3. As shown in FIG. 3, conjugation with the linker 007 significantly prolonged blood circulation of the FAP conjugates as compared to the control group.

TABLE 8
Pharmacokinetic profile of FAP-linker conjugates

| | | | AUCₐₗₗ | |
|---|---|---|---|---|
| | T_{1/2} (hr) | V_{d} (L/kg) | (μg/mL hr) | Cl (L/hr/kg) |
| FAP-DOTAGA | 0.486 ± 0.002 | 0.315 ± 0.020 | 1.56 ± 0.088 | 0.642 ± 0.038 |
| FAP-L007 | 8.10 ± 0.114 | 0.206 ± 0.017 | 45.7 ± 3.23 | 0.022 ± 0.002 |

### Example 5: In Vivo SPECT Imaging of ¹¹¹In-Labeled CLDN18.2 Conjugates

In vivo biodistribution of the CLDN18.2 conjugates was analyzed by SPECT imaging via ¹¹¹In labeling. Briefly, the CLDN18.2 conjugates were labeled by ¹¹¹In at 30° C. for 60 min and purification by NAP-5 column. followed by analysis of radioactive purity through radio-TLC and radio-HPLC on the same day of labelling. The release criteria for radio-labelled sample was ≥90% radiochemical purity.

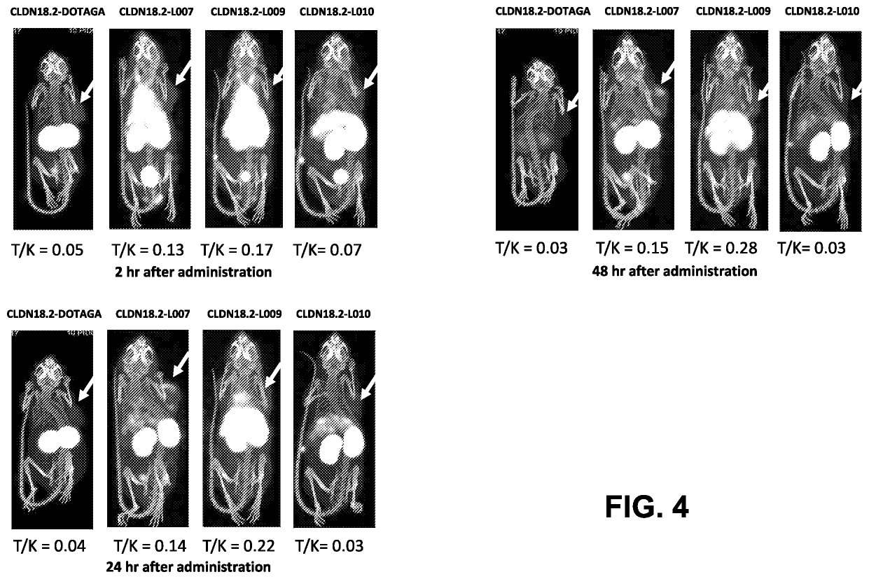

For the imaging study, female athymic nude mice (approximately 6-8 weeks old) were each inoculated with 5×10⁶ CLDN18.2 HEK293T cells via subcutaneous injection into the shoulder. When the tumor volume reached to about 200 to 300 mm³, the animal was injected with the labeled conjugate at a dose of 10 MBq/mouse by a single bolus intravenous injection in tail vein. Animals were then subject to whole-body static SPECT followed by CT at different time points under isoflurane anaesthesia. As shown in FIG. 4, the functional linkers 007 and 009 significantly increased retention of the CLDN18.2 conjugates in tumor over 48 hours as compared to the control group. In addition, the tumor/kidney ratio was increased around 5-10 folds compared to the control group.

### Example 6: In Vivo SPECT Imaging of ¹⁷⁷Lu-Labeled FAP Conjugates

In vivo biodistribution of FAP conjugates was analyzed by SPECT imaging via ¹⁷⁷Lu labeling. Briefly, the FAP conjugates were labeled by ¹⁷⁷Lu with a molar activity of 1 MBq/μg at 37° C. for 60 min and purified by size-exclusion chromatography, followed by analysis of radioactive purity through radio-TLC and radio-HPLC on the same day of labelling. The release criteria for radio-labelled sample was ≥90% radiochemical purity.

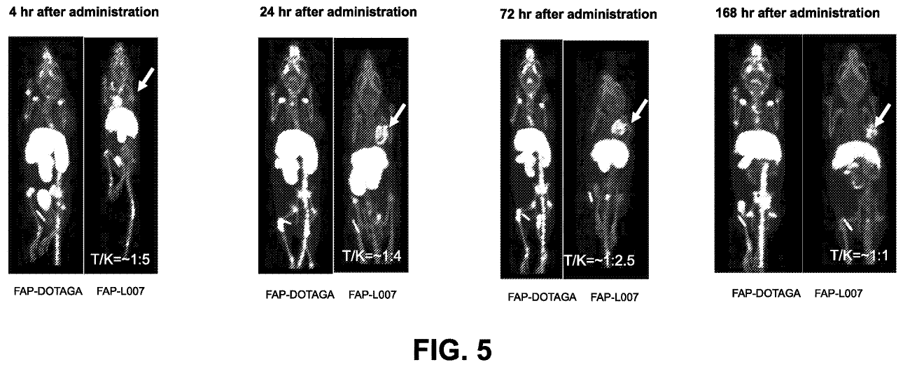

For the imaging study, female athymic nude mice (approximately 6-8 weeks old) were each inoculated with 5×10⁶ FAP expressing HEK293-hFAP cells via subcutaneous injection into the shoulder. When the tumor volume reached to about 150 to 250 mm³, the animal was injected with the labeled conjugate at a dose of 30 MBq/mouse by a single bolus intravenous injection in tail vein. Animals were then subject to whole-body static SPECT followed by CT at different time points under isoflurane anaesthesia. FIG. 5 demonstrates that the functional linker 007 is capable of increasing retention of the FAP conjugates in tumor at different time points. In addition, the functional linker 007 further increased the tumor/kidney (T/K) ratio from 1:5 to 1:1 after 168 hours of administration.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

1. A conjugate comprising:
(a) a single-domain antibody (sdAb) that specifically binds to a tumor antigen;
(b) a drug; and
(c) a functional linker comprising a functional moiety, wherein the functional linker links the drug to the sdAb, wherein the functional linker increases tumor uptake, accumulation, and/or retention of the drug when the conjugate is administered to a subject compared to a conjugate without the functional moiety, and wherein the functional moiety comprises a blood protein binding group.

2-4. (canceled)

5. A conjugate comprising:
(a) an antibody mimetic that specifically binds to a tumor antigen, wherein the antibody mimetic is selected from the group consisting of an Affibody, a DARPin, an Anticalin, an Avimer, a Versabody, and a Duocali;
(b) a drug; and
(c) a functional linker comprising a functional moiety, wherein the functional linker links the drug to the antibody mimetic, and wherein when the conjugate is administered to a subject, compared to a conjugate without the functional moiety, the functional linker is able to: (i) increase tumor uptake, accumulation, and/or retention of the drug, (ii) decrease kidney uptake, accumulation, and/or retention of the drug, (iii) increase tumor/kidney ratio of the drug, (iv) prolong blood circulation of the drug, and/or (v) extend half-life of the drug.

6. The conjugate of claim 1, wherein the functional moiety further comprises one or more components selected from the group consisting of a polyethylene glycol (PEG) group, a renal enzyme cleavable group, a spacer, and any combination thereof.

7. The conjugate of claim 6, wherein the functional moiety comprises a PEG group, wherein the PEG group comprises a structure of 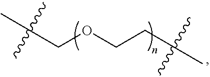
and wherein n is any integer in a range from 1 to 25.

8. (canceled)

9. The conjugate of claim 1, wherein the blood protein binding group is selected from the group consisting of albumin, fetuin, transferrin, and IgG.

10. The conjugate of claim 9, wherein the blood protein binding group comprises an albumin binding group.

11. The conjugate of claim 10, wherein the albumin binding group comprises a structure of 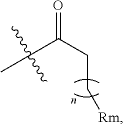
wherein n is any integer ranging from 1 to 20, Rₘ is H, CH₃, or COOH.

12. The conjugate of claim 10, wherein the albumin binding group comprises a structure of 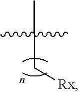
wherein Rₓ is selected from the group consisting of: 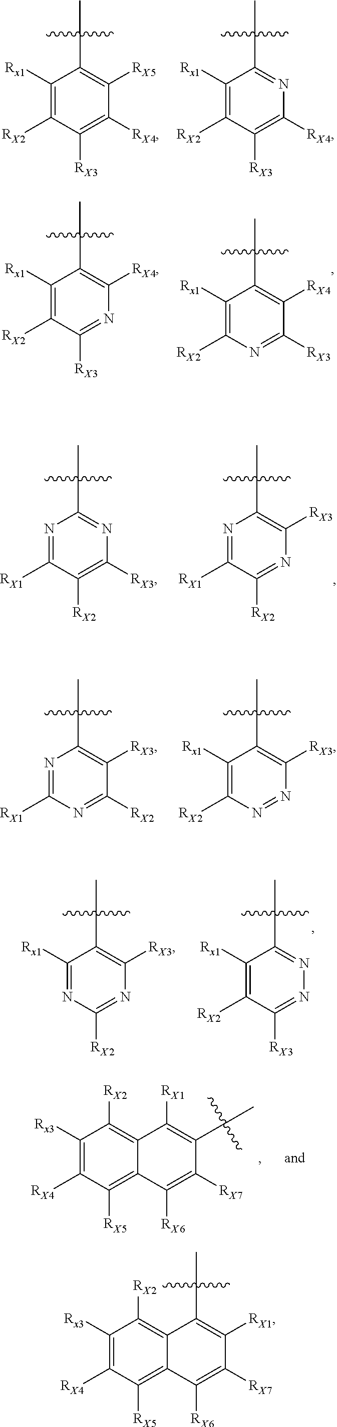
wherein n is any integer ranging from 1 to 6, each of Rₓ₁, Rₓ₂, Rₓ₃, Rₓ₄, Rₓ₅, Rₓ₆, and Rₓ₇ is independently selected from the group consisting of H, N, S, O, Se, P, halogen, a C₅₋₂₀ aryl group, a C₁₋₂₀ alkyl group, a C₂₋₂₀ alkenyl group, and a C₂₋₂₀ alkynyl group, and wherein:
(i) the C₅₋₂₀ aryl group, the C₁₋₂₀ alkyl group, the C₂₋₂₀ alkenyl group, or the C₂₋₂₀ alkynyl group is unsubstituted or substituted by one or more substituents selected from the group consisting of N, S, O, Se, P, and halogen atom;
(ii) 0, 1, or 2 carbon atoms of the C₅₋₂₀ aryl group, the C₁₋₂₀ alkyl group, the C₂₋₂₀ alkenyl group, or the C₂₋₂₀ alkynyl group are replaced by groups selected from the group consisting of C₆₋₁₀ arylene, 5 to 10 membered heteroarylene group, C₃₋₇ carbocyclylen, and 5 to 10 membered heterocyclylene group, and wherein the arylene, heteroarylene, carbocyclylene, and heterocyclylene groups are unsubstituted or substituted by one or more substituents selected from the group consisting of N, S, O, Se, P, halogen, C₁₋₆ alkyl, C₁₋₆ alkoxy, C₁₋₆ alkylthiol, —N(C₁₋₆ alkyl) (C₁₋₆ alkyl), nitro, and sulfonic acid groups; and/or
(iii) 0, 1, or 2-CH— or —CH₂— groups of the C₅₋₂₀ aryl group, the C₁₋₂₀ alkyl group, the C₂₋₂₀ alkenyl group, or the C₂₋₂₀ alkynyl group are replaced by groups selected from the group consisting of —O—, —S—, —S—S—, —C(O)—, and —N(C₁₋₆ alkyl)-group.

13. The conjugate of claim 10, wherein the albumin binding group comprises a structure of 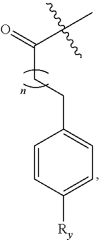
wherein R_{y} is selected from the group consisting of H, F, Cl, Br, I, —CH₃, —OCH₃, COOH, —CF₃, wherein n is any integer ranging from 1 to 6.

14. The conjugate of claim 1, wherein the functional moiety comprises a renal enzyme cleavable group selected from the group consisting of a brush border enzyme cleavable group, a lysosome cleavable enzyme cleavable group, and a combination thereof.

15. (canceled)

16. The conjugate of claim 14, wherein the renal enzyme cleavable group comprises a dipeptide or an oligopeptide, wherein the dipeptide or the oligopeptide comprises a structure selected from the group consisting of methionine-isoleucine, glycine-lysine, glycine-phenylalanine-lysine, methionine-valine-lysine, glycine-tyrosine, glycine-lysine-lysine, glycine-arginine-lysine, aspartic acid-glycine-lysine, methionine-glycine-lysine, methionine-isoleucine-lysine, glycine-tyrosine-lysine, glycine-valine, glycine-isoleucine, methionine-phenylalanine-lysine, glycine-(3-(2-naphthyl)alanine)-lysine, glycine-diphenylalanine-lysine, methionine-glycine-lysine, and any derivative thereof.

17. (canceled)

18. The conjugate of claim 1, wherein the functional linker comprises a spacer comprising a structure selected from the group consisting of a natural amino acid residue, a non-natural amino acid residue, a C₅₋₂₀ aryl group, a C₁₋₂₀ alkyl group, a C₂₋₂₀ alkenyl group, and a C₂₋₂₀ alkynyl group, and wherein:
(i) the C₅₋₂₀ aryl group, the C₁₋₂₀ alkyl group, the C₂₋₂₀ alkenyl group, or the C₂₋₂₀ alkynyl group is unsubstituted or substituted by one or more substituents selected from the group consisting of N, S, O, Se, P, and halogen atom;
(ii) 0, 1, or 2 carbon atoms of the C₅₋₂₀ aryl group, the C₁₋₂₀ alkyl group, the C₂₋₂₀ alkenyl group, or the C₂₋₂₀ alkynyl group are replaced by groups selected from the group consisting of C₆₋₁₀ arylene, 5 to 10 membered heteroarylene group, C₃₋₇ carbocyclylen, and 5 to 10 membered heterocyclylene group, and wherein the arylene, heteroarylene, carbocyclylene, and heterocyclylene groups are unsubstituted or substituted by one or more substituents selected from the group consisting of N, S, O, Se, P, halogen, C₁₋₆ alkyl, C₁₋₆ alkoxy, C₁₋₆ alkylthiol, —N(C₁₋₆ alkyl) (C₁₋₆ alkyl), nitro, and sulfonic acid groups; and/or
(iii) 0, 1, or 2-CH— or —CH₂—groups of the C₁₋₂₀ alkyl group, the C₂₋₂₀ alkenyl group, or the C₂₋₂₀ alkynyl group are replaced by groups selected from the group consisting of —O—, —S—, —S—S—, —C(O)—, and —N(C₁₋₆ alkyl)-groups.

19-20. (canceled)

21. The conjugate of claim 1, wherein the functional linker further comprises a chelator group.

22. (canceled)

23. The conjugate of claim 1, wherein the functional linker is conjugated to the sdAb via an amino acid of the sdAb.

24. (canceled)

25. The conjugate of claim 1, wherein the functional linker comprises a conjugation group capable of conjugating with the sdAb.

26. (canceled)

27. (canceled)

28. The conjugate of claim 1, wherein the tumor antigen is CLDN18.2.

29. The conjugate of claim 28, wherein the sdAb comprises:
a) a CDR1, a CDR2, and a CDR3 as set forth in SEQ ID NO: 1; or
b) a CDR1 comprising an amino acid sequence as set forth in SEQ ID NO: 2, a CDR2
comprising an amino acid sequence as set forth in SEQ ID NO: 3, and a CDR3
comprising an amino acid sequence as set forth in SEQ ID NO: 4.

30. (canceled)

31. The conjugate of claim 1, wherein the tumor antigen is FAP.

32. The conjugate of claim 31, wherein the sdAb comprises:
a) a CDR1, a CDR2, and a CDR3 as set forth in SEQ ID NO: 5; or
b) a CDR1 comprising an amino acid sequence as set forth in SEQ ID NO: 6, a CDR2 comprising an amino acid sequence as set forth in SEQ ID NO: 7, and a CDR3 comprising an amino acid sequence as set forth in SEQ ID NO: 8.

33. (canceled)

34. The conjugate of claim 1, wherein the drug is selected from the group consisting of a chemotherapeutic agent, a toxin, a cytokine, an enzyme, an immunomodulator, a chelating complex, a diagnostic agent, a nanoparticle, and a radioisotope.

35. (canceled)

36. A method for treating a cancer in a subject in need thereof, comprising administering an effective amount of the conjugate of claim 1 to the subject, wherein the cancer expresses a tumor antigen that can be recognized by the conjugate.

37. (canceled)

38. A method of preparing the conjugate of claim 1, comprising conjugating the functional linker and the drug to the sdAb.
